(19) United States
(12) Patent Application Publication
(10) Pub. No.: US 2014/0212415 A1
(43) Pub. Date: Jul. 31, 2014

(54) HYPOXIA-RELATED GENE SIGNATURES FOR CANCER CLASSIFICATION

(71) Applicant: Myriad Genetics, Inc., Salt Lake City, UT (US)

(72) Inventors: Alexander Gutin, Salt Lake City, UT (US); Srikanth Jammulapati, Salt Lake City, UT (US); Susanne Wagner, Salt Lake City, UT (US); Julia Reid, Salt Lake City, UT (US); Darl Flake, Salt Lake City, UT (US)

(21) Appl. No.: 14/172,632

(22) Filed: Feb. 4, 2014

Related U.S. Application Data
(63) Continuation of application No. PCT/US2012/049456, filed on Aug. 3, 2012.
(60) Provisional application No. 61/515,199, filed on Aug. 4, 2011.

(51) Int. Cl. C12Q 1/68

(52) U.S. Cl. CPC C12Q 1/6886
USPC 424/133.1; 506/9; 506/16

(57) ABSTRACT

Biomarkers, particularly hypoxia-related genes, and methods using the biomarkers for molecular classification of disease are provided.

### CROSS-REFERENCE TO RELATED APPLICATIONS

The present application is a continuation application of International Application Serial No. PCT/US2012/049456, filed 3 Aug. 2012 and published 7 Feb. 2013 as WO/2013/020019A9. The present application and International Application Serial No. PCT/US2012/049456 are related to and claim the priority benefit of U.S. provisional patent application Ser. No. 61/515,199, filed 4 Aug. 2011. Each of these applications is incorporated herein by reference in its entirety.

### FIELD OF THE INVENTION

The invention generally relates to molecular classification of cancer using hypoxia-related biomarkers.

### BACKGROUND OF THE INVENTION

Cancer is a major public health problem, accounting for nearly one out of every four deaths in the United States. American Cancer Society, Facts and Figures 2010. Patient prognosis generally improves with earlier detection of cancer. Indeed, more readily detectable cancers such as breast cancer have a substantially better survival rate than cancers that are more difficult to detect (e.g., ovarian cancer).

Though many treatments have been devised for various cancers, these treatments often vary in severity of side effects. It is useful for clinicians to know how aggressive a patient's cancer is in order to determine how aggressively to treat the cancer.

Some tools have been devised to help physicians in deciding which patients need aggressive treatment and which do not. In fact, several clinical parameters are currently in use for this purpose in various different cancers. Despite these advances, however, many patients are given improper cancer treatments and there is still a serious need for novel and improved tools for predicting cancer recurrence.

### SUMMARY OF THE INVENTION

The present invention is based in part on the discovery that hypoxia-related genes or HRGs (genes where changes in expression are induced by the cellular condition hypoxia) are particularly powerful genes for classifying cancers (especially lung and colon cancers).

Accordingly, in a first aspect of the present invention, a method is provided for determining gene expression in a tumor sample from a patient identified as having lung cancer or colon (including colorectal) cancer. Generally, the method includes at least the following steps: (1) providing (or obtaining) a tumor sample from a patient identified as having lung cancer or colon (including colorectal) cancer; (2) determining the expression of a panel of biomarkers in said tumor sample including at least 5 HRGs; and (3) providing a test value by (a) weighting the determined expression of each of a plurality of test genes selected from said panel of biomarkers with a predefined coefficient, and (b) combining the weighted expression to provide said test value, wherein the combined weight given to said at least 5 HRGs is at least 40% (or 50%, 60%, 70%, 80%, 90%, 95% or 100%) of the total weight given to the expression of all of said plurality of test genes. In some embodiments at least 50%, at least 75% or at least 90% of said plurality of test genes are HRGs.

In some embodiments the invention provides a method of determining gene expression in a tumor sample from a patient identified as having lung cancer or colon cancer, comprising: (1) providing (or obtaining) a tumor sample from a patient identified as having lung cancer or colon (including colorectal) cancer; (2) determining the expression levels of at least 5 hypoxia-related genes in said tumor sample; and (3) providing a test value reflecting the overall expression level of said at least 5 hypoxia-related genes in said tumor sample.

In some embodiments the determining step comprises: measuring the amount of mRNA in said tumor sample transcribed from each of between 5 and 200 HRGs; and measuring the amount of mRNA of one or more housekeeping genes in said tumor sample. Measuring mRNA may include measuring DNA reverse transcribed from mRNA.

In some embodiments, the plurality of test genes comprises at least 6 HRGs, or at least 7, 8, 9, 10, 15, 20, 25 or 30 HRGs. Preferably, all of the test genes are HRGs. In some embodiments of this and all other aspects of the invention, the plurality of test genes comprises at least 6 HRGs, or at least 7, 8, 9, 10, 15, 20, 25 or 30 of the HRGs listed in Table 1 and/or Table 2. In some embodiments the plurality of test genes comprises all the HRGs listed in Table 1 and/or Table 2.

In another aspect of the present invention, a method is provided for determining the prognosis of lung cancer or colon cancer, which comprises determining in a tumor sample (e.g., from a patient identified as having lung cancer or colon cancer), the expression of at least 6, 8 or 10 HRGs, wherein overexpression of said at least 6, 8 or 10 HRGs indicates a poor prognosis or an increased likelihood of recurrence of cancer in the patient. In some embodiments of this and all other aspects of the invention the tumor sample is from a patient identified as having lung cancer or colon cancer.

In one embodiment, the prognosis method comprises (1) determining in a tumor sample the expression of a panel of biomarkers in said tumor sample including at least 4 or at least 8 HRGs; (2) providing a test value by (a) weighting the determined expression of each of a plurality of test genes selected from the panel of biomarkers with a predefined coefficient, and (b) combining the weighted expression to provide the test value, wherein the combined weight given to said at least 4 or at least 8 HRGs is at least 40% (or 50%, 60%, 70%, 80%, 90%, 95% or 100%) of the total weight given to the expression of all of said plurality of test genes; and (3) correlating an increased level (e.g., overall) of expression of the plurality of test genes to a poor prognosis or a high likelihood of disease progression or recurrence of cancer. In some embodiments at least 50%, at least 75% or at least 90% of said plurality of test genes are HRGs. In some embodiments, if there is no increase (e.g., overall) in the expression of the test genes, it would indicate a good prognosis or a low likelihood of disease progression or recurrence of cancer in the patient.

In some embodiments, the prognosis method further includes a step of comparing the test value provided in step (2) above to one or more reference values, and correlating the test value to a risk of cancer progression or risk of cancer recurrence. Optionally an increased likelihood of poor prognosis is indicated if the test value is greater than the reference value.

In yet another aspect, the present invention also provides a method of treating cancer in a patient, comprising: (1) determining in a tumor sample from a patient the expression of a panel of biomarkers in the tumor sample including at least 4 or at least 8 HRGs; (2) providing a test value by (a) weighting the determined expression of each of a plurality of test genes selected from said panel of biomarkers with a predefined coefficient, and (b) combining the weighted expression to provide the test value, wherein the combined weight given to said at least 4 or at least 8 HRGs is at least 40% (or 50%, 60%, 70%, 80%, 90%, 95% or 100%) of the total weight given to the expression of all of said plurality of test genes; (3) correlating an increased level of expression of the plurality of test genes to a poor prognosis, or a low (or not increased) level of expression of the plurality of test genes to a good prognosis; and (4) recommending, prescribing or administering a treatment regimen or watchful waiting based at least in part on the prognosis provided in step (3). In some embodiments at least 50%, at least 75% or at least 90% of said plurality of test genes are HRGs.

The present invention further provides a diagnostic kit useful in the above methods, the kit generally comprising, in a compartmentalized container, a plurality of oligonucleotides hybridizing to at least 8 test genes (or gene products), wherein less than 10%, 30% or less than 40% of all of the at least 8 test genes are non-HRGs; and one or more oligonucleotides hybridizing to at least one housekeeping gene. In another embodiment the invention provides a diagnostic kit for prognosing cancer in a patient comprising the above components. In another embodiment the invention provides the use of a diagnostic kit comprising the above components for prognosing cancer in a patient. The oligonucleotides can be hybridizing probes for hybridization with the test genes under stringent conditions or primers suitable for PCR amplification of the test genes. In one embodiment, the kit consists essentially of, in a compartmentalized container, a first plurality of PCR reaction mixtures for PCR amplification of from 5 or 10 to about 300 test genes, wherein at least 25%, at least 50%, at least 60% or at least 80% of such test genes are HRGs, and wherein each reaction mixture comprises a PCR primer pair for PCR amplifying one of the test genes; and a second plurality of PCR reaction mixtures for PCR amplification of at least one housekeeping gene.

The present invention also provides the use of (1) a plurality of oligonucleotides hybridizing to at least 4 or at least 8 HRGs; and (2) one or more oligonucleotides hybridizing to at least one housekeeping gene, for the manufacture of a diagnostic product. In another embodiment the diagnostic product is for determining the expression of the test genes in a tumor sample from a patient, to predict the prognosis of cancer, wherein an increased level of the overall expression of the test genes indicates a poor prognosis or an increased likelihood of recurrence of cancer in the patient, whereas if there is no increase in the overall expression of the test genes, it would indicate a good prognosis or a low likelihood of recurrence of cancer in the patient. In some embodiments, the oligonucleotides are PCR primers suitable for PCR amplification of the test genes. In other embodiments, the oligonucleotides are probes hybridizing to the test genes under stringent conditions. In some embodiments, the plurality of oligonucleotides are probes for hybridization under stringent conditions to, or are suitable for PCR amplification of, from 4 to about 300 test genes, at least 50%, 70% or 80% or 90% of the test genes being HRGs. In some other embodiments, the plurality of oligonucleotides are hybridization probes for, or are suitable for PCR amplification of, from 20 to about 300 test genes, at least 30%, 40%, 50%, 70% or 80% or 90% of the test genes being HRGs.

The present invention further provides systems related to the above methods of the invention. In one embodiment the invention provides a system for determining gene expression in a tumor sample, comprising: (1) a sample analyzer for determining the status of a panel of biomarkers in a sample including at least 4 HRGs, wherein the sample analyzer contains the sample, mRNA from the sample and expressed from the genes in the panel of biomarkers, or DNA reverse transcribed from said mRNA; (2) a first computer program for (a) receiving gene expression data on at least 4 test genes selected from the panel of biomarkers, (b) weighting the determined expression of each of the test genes with a predefined coefficient, and (c) combining the weighted expression to provide a test value, wherein at least 50%, 70%, 80%, or 90% of the at least 4 test genes are HRGs; and optionally (3) a second computer program for comparing the test value to one or more reference values each associated with a predetermined degree of risk of cancer. In some embodiments the combined weight given to the HRGs is at least 40% (or 50%, 60%, 70%, 80%, 90%, 95% or 100%) of the total weight given to the expression of all of the plurality of test genes.

In another embodiment the invention provides a system for determining gene expression in a tumor sample, comprising: (1) a sample analyzer for determining the status of a panel of biomarkers in a tumor sample including at least 4 HRGs, wherein the sample analyzer contains the tumor sample which is from a patient identified as having lung cancer or colon cancer, mRNA expressed from the genes in the panel of biomarkers, or DNA reverse transcribed from such mRNA; (2) a first computer program for (a) receiving gene expression data on at least 4 test genes selected from the panel of biomarkers, (b) weighting the determined expression of each of the test genes with a predefined coefficient, and (c) combining the weighted expression to provide a test value, wherein at least 50%, 70%, 80%, or 90% of at least 4 test genes are HRGs; and optionally (3) a second computer program for comparing the test value to one or more reference values each associated with a predetermined degree of risk of cancer recurrence or progression of lung cancer or colon cancer. In some embodiments, the system further comprises a display module displaying the comparison between the test value and the one or more reference values, or displaying a result of the comparing step. In some embodiments the combined weight given to the HRGs is at least 40% (or 50%, 60%, 70%, 80%, 90%, 95% or 100%) of the total weight given to the expression of all of the plurality of test genes.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following Detailed Description, and from the Claims.

### BRIEF DESCRIPTION OF THE DRAWINGS

FIG. 1 shows a Kaplan-Meier plot of disease-free survival versus stage in colorectal cancer samples.

FIG. 2 shows a Kaplan-Meier plot of disease-free survival versus hypoxia expression in stage II colorectal cancer samples (based on hypoxia score).

FIG. 3 is an illustration of a computer system of the invention.

FIG. 4 is an illustration of a computer-implemented method of the invention.

FIG. 5 shows a Kaplan-Meier plot of progression-free survival in colorectal cancer samples.

FIG. 6 shows the distribution of hypoxia scores for colorectal samples.

FIG. 7 shows a Kaplan-Meier plot of progression-free survival in colorectal cancer samples.

FIG. 8 illustrates the correlation of the expression of various HRGs to each other.

FIG. 9 shows univariate tests for various HRGs with the three outcome measures in lung samples as well as the HRGs' correlation to two different HRG means.

FIG. 10 shows a distribution of recurrences amongst colorectal cancer patients in Example 4.

FIG. 11 shows Kaplan-Meier plots of recurrence-free survival and overall survival in colorectal cancer samples.

FIG. 12 illustrates the correlation between HRG overexpression recurrence amongst adjuvant and non-adjuvant colorectal cancer patients.

### DETAILED DESCRIPTION OF THE INVENTION

### I. Determining Hypoxia-Related Gene Expression

The present invention is based in part on the discovery that hypoxia-related genes are particularly powerful genes for classifying colon cancer. “Hypoxia-related gene” and “HRG” herein refer to a gene where changes in expression level are induced by the cellular condition hypoxia (i.e., low cellular levels of oxygen). Often HRGs have clear, recognized hypoxia-related function. However, some HRGs have expression variations induced by hypoxia without having a clear, direct role in the hypoxia response. Thus an HRG according to the present invention need not have a recognized role in the hypoxia response.

Whether a particular gene is a hypoxia-related gene may be determined by any technique known in the art, including those taught in Lal et al., J. NATL. CANCER INST. (2001) 93:1337-1343; Leonard et al., J. BIOL. CHEM. (2003) 278:40296-40304. For example, cell lines may be grown with the use of standard cell culture techniques either in equilibrium with atmospheric oxygen or in an Environmental Chamber with reduced oxygen designed to approximate the tumor hypoxia levels, see, e.g., Dewhirst et al., RADIAT. RES. (1992) 130:171-182, for hypoxic conditions. The expression level of any test gene (or any group of genes) may then be determined by any known technique (e.g., quantitative (including real-time) PCR, microarray, etc.) in both the standard oxygen and hypoxia cultures. These expression levels may then be compared and any genes showing a significant difference, see, e.g., Lal et al. (2001), at 1337 (“Statistical Analysis”), between the standard oxygen and hypoxia cultures may be deemed hypoxia-related genes. Whether a gene is hypoxia-related may be confirmed by a variety of assays, including testing to see if the gene is regulated by HIF-1 (e.g., the subunit HIF-1α). See, e.g., Lal et al. (2001), at 1337 (“HIF-1 Transfection”); id. at 1340. Exemplary HRGs are listed in Tables 1 & 2 below.

TABLE 1

| Gene | Entrez |
|---|---|
| Symbol | GeneId |
| ADFP | 123 |
| ADM | 133 |
| ADORA2B | 136 |
| ALDOA | 226 |
| ALDOC | 230 |
| ANGPTL4 | 51129 |
| APOBEC3C | 27350 |
| BHLHB2 | 8553 |
| BNIP3 | 664 |
| BNIP3L | 665 |
| C10orf10 | 11067 |
| C3orf28 | 26355 |
| CA9 | 768 |
| DDIT4 | 54541 |
| DUSP1 | 1843 |
| EGFR | 1956 |
| EGLN3 | 112399 |
| ENO2 | 2026 |
| ERO1L | 30001 |
| ERRFI1 | 54206 |
| FAM13A1 | 10144 |
| FBXO44 | 93611 |
| FOS | 2353 |
| FOSL2 | 2355 |
| GAPDH | 2597 |
| GJA1 | 2697 |
| GNB2L1 | 10399 |
| GYS1 | 2997 |
| HIG2 | 29923 |
| HIST1H1C | 3006 |
| HIST2H2BE | 8349 |
| HLA-DRB3 | 3125 |
| HMGCL | 3155 |
| HOXA13 | 3209 |
| HSPA5 | 3309 |
| IGF2 | 3481 |
| IGFBP3 | 3486 |
| IGFBP5 | 3488 |
| INHA | 3623 |
| INHBB | 3625 |
| ITPR1 | 3708 |
| JMJD6 | 23210 |
| LDHA | 3939 |
| LOX | 4015 |
| LOXL2 | 4017 |
| MIF | 4282 |
| MXI1 | 4601 |
| NDRG1 | 10397 |
| NR3C1 | 2908 |
| NRN1 | 51299 |
| P4HA1 | 5033 |
| P4HA2 | 8974 |
| PDGFB | 5155 |
| PDK1 | 5163 |
| PFKFB3 | 5209 |
| PFKFB4 | 5210 |
| PFKP | 5214 |
| PGK1 | 5230 |
| PLOD2 | 5352 |
| PPP1R3C | 5507 |
| PROX1 | 5629 |
| RASGRP1 | 10125 |
| RNASE4 | 6038 |
| SAT1 | 6303 |
| SERPINE1 | 5054 |
| SERPINI1 | 5274 |
| SLC16A3 | 9123 |
| SLC2A1 | 6513 |
| SLC2A3 | 6515 |
| SLC6A8 | 6535 |
| SOX9 | 6662 |
| SPAG4 | 6676 |
| SSR4 | 6748 |
| STC1 | 6781 |
| STC2 | 8614 |
| TFF1 | 7031 |
| TMEM45A | 55076 |
| TNC | 3371 |
| TPI1 | 7167 |
| VEGFA | 7422 |
| ZFP36 | 7538 |
| ZFP36L2 | 678 |
| ZNF395 | 55893 |

TABLE 2

| Gene Symbol | Entrez GeneId |
|---|---|
| ADM | 133 |
| ALDOA | 226 |
| ALDOC | 230 |
| ANGPTL4 | 51129 |
| BHLHB2 | 8553 |
| BNIP3 | 664 |
| DDIT4 | 54541 |
| ENO2 | 2026 |
| ERO1L | 30001 |
| GAPDH | 2597 |
| GYS1 | 2997 |
| IGFBP3 | 3486 |
| IGFBP5 | 3488 |
| ITPR1 | 3708 |
| LDHA | 3939 |
| LOX | 4015 |
| LOXL2 | 4017 |
| MIF | 4282 |
| MXI1 | 4601 |
| NDRG1 | 10397 |
| P4HA1 | 5033 |
| P4HA2 | 8974 |
| PDGFB | 5155 |
| PDK1 | 5163 |
| PFKP | 5214 |
| PGK1 | 5230 |
| PLOD2 | 5352 |
| PPP1R3C | 5507 |
| PROX1 | 5629 |
| SERPINE1 | 5054 |
| SLC16A3 | 9123 |
| SLC2A1 | 6513 |
| SLC2A3 | 6515 |
| STC2 | 8614 |
| TNC | 3371 |
| TPI1 | 7167 |
| VEGFA | 7422 |

Accordingly, in a first aspect of the present invention, a method is provided for determining gene expression in a sample. Generally, the method includes at least the following steps: (1) obtaining a sample from a patient; (2) determining the expression of a panel of biomarkers in the sample including at least 2, 4, 6, 8 or 10 HRGs; and (3) providing a test value by (a) weighting the determined expression of each of a plurality of test genes selected from said panel of biomarkers with a predefined coefficient, and (b) combining the weighted expression to provide said test value, wherein the combined weight given to said at least 4 or 5 or 6 HRGs is at least 40% (or 50%, 60%, 70%, 80%, 90%, 95% or 100%) of the total weight given to the expression of all of said plurality of test genes. In some embodiments at least 20%, 50%, 75%, or 90% of said plurality of test genes are HRGs.

In some embodiments, said plurality of test genes comprises at least 2, 3, 4, 5, 6, 7, 8, 9, 10, 12, 14, 16, 18, 20, 25, 30, 35, 40, 45, 50, 60, 70, 80, 90, or 100 or more HRGs. In some embodiments, said plurality of test genes comprises at least 2, 3, 4, 5, 6, 7, 8, 9, 10, 12, 14, 16, 18, 20, 25, 30, 35, 40, 45, 50, 60, 70, or 80 or more HRGs selected from Tables 1, 2, 3, 5, 6, 7, 10, 11, 12, 13, 14, 15, 16, 17, 18, 19, 20, 21, 22, or 23. In some embodiments, said plurality of test genes comprises at least 2 HRGs, and the combined weight given to said at least 2 HRGs is at least 40% (or 50%, 60%, 70%, 80%, 90%, 95% or 100%) of the total weight given to the expression of all of said plurality of test genes. In some embodiments, said plurality of test genes comprises at least 4 or 5 or 6 HRGs, and the combined weight given to said at least 4 or 5 or 6 HRGs is at least 40% (or 50%, 60%, 70%, 80%, 90%, 95% or 100%) of the total weight given to the expression of all of said plurality of test genes. The meaning of this percentage of total weight is explained further below.

In some embodiments, said plurality of test genes comprises one or more HRGs constituting from 1% to about 95% of said plurality of test genes, and the combined weight given to said one or more HRGs is at least 40%, 50%, 60%, 70%, 80%, 90%, 95% or 100% of the total weight given to the expression of all of said plurality of test genes. Preferably, said plurality of test genes includes at least 2, preferably 4, more preferably at least 5 HRGs, and most preferably at least 6 HRGs.

The sample used in the method may be a sample derived from the lung, colon or rectum, e.g., by way of biopsy or surgery. The sample may also be cells shed by the lung, colon or rectum, e.g., into blood, urine, sputum, feces, etc. Samples from an individual diagnosed with cancer may be used for the cancer prognosis in accordance with the present invention. Unless otherwise indicated, “obtaining a sample” herein means “providing or obtaining.”

For example, the method may be performed on a tumor sample from a patient identified as having lung cancer or colon cancer. As used herein, “colon cancer” and “colorectal cancer” are used interchangeably to refer to colorectal cancer. Such a method includes at least the following steps: (1) obtaining a tumor sample from a patient identified as having lung cancer or colon cancer; (2) determining the expression of a panel of biomarkers in the tumor sample including at least 2, 4, 6, 8 or 10 HRGs; and (3) providing a test value by (a) weighting the determined expression of each of a plurality of test genes selected from said panel of biomarkers with a predefined coefficient, and (b) combining the weighted expression to provide said test value, wherein the combined weight given to said at least 4 or 5 or 6 HRGs is at least 40% (or 50%, 60%, 70%, 80%, 90%, 95% or 100%) of the total weight given to the expression of all of said plurality of test genes. In some embodiments at least 20%, 50%, 75%, or 90% of said plurality of test genes are HRGs.

The method also may be performed on a sample from a patient who has not been diagnosed with (but may be suspected of having) lung cancer or colon cancer. The sample may be a tissue biopsy or surgical sample directly from the organ of lung, colon or rectum, or cells shedded from such an organ in a bodily fluid (e.g., blood or urine) or other bodily sample (e.g., feces). Such a method includes at least the following steps: (1) obtaining a sample that is a tissue or cell from the lung, colon or rectum of an individual who has not been diagnosed of cancer; (2) determining the expression of a panel of biomarkers in the sample including at least 2, 4, 6, 8 or 10 HRGs; and (3) providing a test value by (a) weighting the determined expression of each of a plurality of test genes selected from said panel of biomarkers with a predefined coefficient, and (b) combining the weighted expression to provide said test value, wherein the combined weight given to said at least 4 or 5 or 6 HRGs is at least 40% (or 50%, 60%, 70%, 80%, 90%, 95% or 100%) of the total weight given to the expression of all of said plurality of test genes. In some embodiments at least 20%, 50%, 75%, or 90% of said plurality of test genes are HRGs.

In some embodiments of the method in accordance with this aspect of the invention, said plurality of test genes includes at least 2 HRGs which constitute at least 50% or at least 60% of said plurality of test genes. In some embodiments, said plurality of test genes includes at least 4 HRGs which constitute at least 20% or 30% or 50% or 60% of said plurality of test genes.

In some embodiments, said plurality of test genes includes the HRGs INHBA and FAP. In some embodiments, the sample is from prostate, lung, bladder or brain, but not from breast, and said panel of biomarkers in the method described above comprises INHBA and FAP, and said plurality of test genes includes INHBA and FAP, and optionally the weighting of the expression of the test genes is according to that in O'Connell et al., J. CLIN. ONCOL. (2010) 28:3937-3944, which is incorporated herein by reference.

In some embodiments the plurality of test genes (or panel) include less than some specific number or proportion of cell-cycle progression genes. As used herein, “cell-cycle progression gene” and “CCP gene” mean a gene whose expression level closely tracks the progression of the cell through the cell-cycle. See, e.g., Whitfield et al., MOL. BIOL. CELL (2002) 13:1977-2000. More specifically, CCP genes show periodic increases and decreases in expression that coincide with certain phases of the cell cycle—e.g., STK15 and PLK show peak expression at G2/M. Id. Often CCP genes have clear, recognized cell-cycle related function. However, some CCP genes have expression levels that track the cell-cycle without having an obvious, direct role in the cell-cycle. Thus a CCP gene according to the present invention need not have a recognized role in the cell-cycle. Exemplary CCP genes include ANLN (Entrez Geneld no. 54443), C20orf20 (Entrez Geneld no. 55257), MRPS17 (Entrez Geneld no. 51373), NME1 (Entrez Geneld no. 4830), CDCA4 (Entrez Geneld no. 55038), EIF2S1 (Entrez Geneld no. 1965), PSMA7 (Entrez Geneld no. 5688), PSMB7 (Entrez Geneld no. 5695), PSMD2 (Entrez Geneld no. 5708), ACOT7 (Entrez Geneld no. 11332), MRPL15 (Entrez Geneld no. 29088), CDKN3 (Entrez Geneld no. 1033), MRPL13 (Entrez Geneld no. 28998), SHCBP1 (Entrez Geneld no. 79801), TUBA1B (Entrez Geneld no. 10376), CTSL2 (Entrez Geneld no. 1515), PSRC1 (Entrez Geneld no. 84722), KIF4A (Entrez Geneld no. 24137), and TUBA1C (Entrez Geneld no. 84790). In some embodiments the plurality of test genes includes less than 10%, 9%, 8%, 7%, 6%, 5%, 4%, 3%, 2%, or 1% CCP genes. In one embodiment the plurality of test genes includes no CCP genes.

In the various embodiments described above where the plurality of test genes includes other than HRGs, preferably the weight coefficient given to each HRG in said plurality of test genes is greater than 1/N where N is the total number of test genes in the plurality of test genes.

In another aspect of the present invention, a method is provided for analyzing gene expression in a sample. Generally, the method includes at least the following steps: (1) obtaining expression level data from a sample for a panel of biomarkers including at least 2, 4, 6, 8 or 10 HRGs; and (2) providing a test value by (a) weighting the determined expression of each of a plurality of test genes selected from said panel of biomarkers with a predefined coefficient, and (b) combining the weighted expression to provide said test value, wherein the combined weight given to said at least 4 or 5 or 6 HRGs is at least 40% (or 50%, 60%, 70%, 80%, 90%, 95% or 100%) of the total weight given to the expression of all of said plurality of test genes. In some embodiments at least 20%, 50%, 75%, or 90% of said plurality of test genes are HRGs. In some embodiments, the plurality of test genes includes at least 6 HRGs, which constitute at least 35%, 50% or 75% of said plurality of test genes. In some embodiments, the plurality of test genes includes at least 8 HRGs, which constitute at least 20%, 35%, 50% or 75% of said plurality of test genes. In some embodiments the expression level data comes from a tumor sample from a patient identified as having prostate cancer, lung cancer, bladder cancer or brain cancer.

Gene expression can be determined either at the RNA level (i.e., noncoding RNA (ncRNA), mRNA, miRNA, tRNA, rRNA, snoRNA, siRNA, or piRNA) or at the protein level. Unless otherwise indicated explicitly or as would be clear in context to one skilled in the art, references herein to RNA (including measuring RNA expression or levels) include DNA reverse transcribed from such RNA. Levels of proteins in a tumor sample can be determined by any known techniques in the art, e.g., HPLC, mass spectrometry, or using antibodies specific to selected proteins (e.g., IHC, ELISA, etc.).

In a some embodiment, the amount of RNA transcribed from the panel of biomarkers including test genes in the sample is measured. In addition, the amount of RNA of one or more housekeeping genes in the sample is also measured, and used to normalize or calibrate the expression of the test genes. The terms “normalizing genes” and “housekeeping genes” are defined herein below.

In some embodiments, the plurality of test genes includes at least 2, 3 or 4 HRGs, which constitute at least 50%, 75% or 80% of the plurality of test genes, and preferably 100% of the plurality of test genes. In some embodiments, the plurality of test genes includes at least 5, 6 or 7, or at least 8 HRGs, which constitute at least 20%, 25%, 30%, 40%, 50%, 60%, 70%, 75%, 80% or 90% of the plurality of test genes, and preferably 100% of the plurality of test genes.

In some other embodiments, the plurality of test genes includes at least 8, 10, 12, 15, 20, 25 or 30 HRGs, which constitute at least 20%, 25%, 30%, 40%, 50%, 60%, 70%, 75%, 80% or 90% of the plurality of test genes, and preferably 100% of the plurality of test genes.

As will be apparent to a skilled artisan apprised of the present invention and the disclosure herein, “tumor sample” means any biological sample containing one or more tumor cells, or one or more tumor derived RNA or protein, and obtained from a cancer patient. For example, a tissue sample obtained from a tumor tissue of a cancer patient is a useful tumor sample in the present invention. The tissue sample can be an FFPE sample, or fresh frozen sample, and preferably contain largely tumor cells. A single malignant cell from a cancer patient's tumor is also a useful tumor sample. Such a malignant cell can be obtained directly from the patient's tumor, or purified from the patient's bodily fluid or waste such as blood, urine, or feces. In addition, a bodily sample such as blood, urine, sputum, saliva, or feces containing one or tumor cells, or tumor-derived RNA or proteins, can also be useful as a tumor sample for purposes of practicing the present invention.

Those skilled in the art are familiar with various techniques for determining the status of a gene or protein in a tissue or cell sample including, but not limited to, microarray analysis (e.g., for assaying mRNA or microRNA expression, copy number, etc.), quantitative real-time PCR™ (“qRT-PCR™”, e.g., TaqMan™), immunoanalysis (e.g., ELISA, immunohistochemistry), etc. The activity level of a polypeptide encoded by a gene may be used in much the same way as the expression level of the gene or polypeptide. Often higher activity levels indicate higher expression levels while lower activity levels indicate lower expression levels. Thus, in some embodiments, the invention provides any of the methods discussed above, wherein the activity level of a polypeptide encoded by the HRG is determined rather than or in addition to the expression level of the HRG. Those skilled in the art are familiar with techniques for measuring the activity of various such proteins, including those encoded by the genes listed in Tables 1, 2, 3, 5, 6, 7, 10, 11, 12, 13, 14, 15, 16, 17, 18, 19, 20, 21, 22, or 23. The methods of the invention may be practiced independent of the particular technique used.

In some embodiments, the expression of one or more normalizing genes is also obtained for use in normalizing the expression of test genes. As used herein, “normalizing genes” referred to the genes whose expression is used to calibrate or normalize the measured expression of the gene of interest (e.g., test genes). Importantly, the expression of normalizing genes should be independent of cancer outcome/prognosis, and the expression of the normalizing genes is very similar among all the tumor samples. The normalization ensures accurate comparison of expression of a test gene between different samples. For this purpose, housekeeping genes known in the art can be used. Housekeeping genes are well known in the art, with examples including, but are not limited to, GUSB (glucuronidase, beta), HMBS (hydroxymethylbilane synthase), SDHA (succinate dehydrogenase complex, subunit A, flavoprotein), UBC (ubiquitin C) and YWHAZ (tyrosine 3-monooxygenase/tryptophan 5-monooxygenase activation protein, zeta polypeptide). One or more housekeeping genes can be used. Preferably, at least 2, 5, 10 or 15 housekeeping genes are used to provide a combined normalizing gene set. The amount of gene expression of such normalizing genes can be averaged, combined together by straight additions or by a defined algorithm. Some examples of particularly useful housekeeping genes for use in the methods and compositions of the invention include those listed in Table A below.

TABLE A

| Gene | Entrez | Applied Biosystems | RefSeq |
|---|---|---|---|
| Symbol | GeneID | Assay ID | Accession Nos. |
| CLTC* | 1213 | Hs00191535_m1 | NM_004859.3 |
| GUSB | 2990 | Hs99999908_m1 | NM_000181.2 |
| HMBS | 3145 | Hs00609297_m1 | NM_000190.3 |
| MMADHC* | 27249 | Hs00739517_g1 | NM_015702.2 |
| MRFAP1* | 93621 | Hs00738144_g1 | NM_033296.1 |
| PPP2CA* | 5515 | Hs00427259_m1 | NM_002715.2 |
| PSMA1* | 5682 | Hs00267631_m1 | |
| PSMC1* | 5700 | Hs02386942_g1 | NM_002802.2 |
| RPL13A* | 23521 | Hs03043885_g1 | NM_012423.2 |
| RPL37* | 6167 | Hs02340038_g1 | NM_000997.4 |
| RPL38* | 6169 | Hs00605263_g1 | NM_000999.3 |
| RPL4* | 6124 | Hs03044647_g1 | NM_000968.2 |
| RPL8* | 6132 | Hs00361285_g1 | NM_033301.1; |
| | | | NM_000973.3 |
| RPS29* | 6235 | Hs03004310_g1 | NM_001030001.1; |
| | | | NM_001032.3 |
| SDHA | 6389 | Hs00188166_m1 | NM_004168.2 |
| SLC25A3* | 6515 | Hs00358082_m1 | NM_213611.1; |
| | | | NM_002635.2; |
| | | | NM_005888.2 |
| TXNL1* | 9352 | Hs00355488_m1 | NR_024546.1; |
| | | | NM_004786.2 |
| UBA52* | 7311 | Hs03004332_g1 | NM_001033930.1; |
| | | | NM_003333.3 |
| UBC | 7316 | Hs00824723_m1 | NM_021009.4 |
| YWHAZ | 7534 | Hs00237047_m1 | NM_003406.3 |

In the case of measuring RNA levels for the genes, one convenient and sensitive approach is real-time quantitative PCR™ (qPCR) assay, following a reverse transcription reaction. Typically, a cycle threshold (Cₜ) is determined for each test gene and each normalizing gene, i.e., the number of cycle at which the fluoescence from a qPCR reaction above background is detectable.

The overall expression of the one or more normalizing genes can be represented by a “normalizing value” which can be generated by combining the expression of all normalizing genes, either weighted equally (straight addition or averaging) or by different predefined coefficients. For example, in one simple manner, the normalizing value C_{tH} can be the cycle threshold (Cₜ) of one single normalizing gene, or an average of the Cₜ values of 2 or more, preferably 10 or more, or 15 or more normalizing genes, in which case, the predefined coefficient is 1/N, where N is the total number of normalizing genes used. Thus, C_{tH}=(C_{tH1}+C_{tH2}+ . . . C_{tHn})/N. As will be apparent to skilled artisans, depending on the normalizing genes used, and the weight desired to be given to each normalizing gene, any coefficients (from 0/N to N/N) can be given to the normalizing genes in weighting the expression of such normalizing genes. That is, C_{tH}=xC_{tH1}+yC_{tH2}+ . . . zC_{tHn}, wherein x+y+ . . . +z=1.

As discussed above, the methods of the invention generally involve determining the level of expression of a panel of HRGs. With modern high-throughput techniques, it is often possible to determine the expression level of tens, hundreds or thousands of genes. Indeed, it is possible to determine the level of expression of the entire transcriptome (i.e., each transcribed gene in the genome). Once such a global assay has been performed, one may then informatically analyze one or more subsets (i.e., panels) of genes. After measuring the expression of hundreds or thousands of genes in a sample, for example, one may analyze (e.g., informatically) the expression of a panel comprising primarily HRGs according to the present invention by combining the expression level values of the individual test genes to obtain a test value.

As will be apparent to a skilled artisan, the test value provided in the present invention represents the overall expression level of the plurality of test genes composed of substantially HRGs. In one embodiment, to provide a test value in the methods of the invention, the normalized expression for a test gene can be obtained by normalizing the measured Cₜ for the test gene against the C_{tH}, i.e., ΔCₜ₁=(Cₜ₁−C_{tH}). Thus, the test value representing the overall expression of the plurality of test genes can be provided by combining the normalized expression of all test genes, either by straight addition or averaging (i.e., weighted equally) or by a different predefined coefficient. For example, the simplest approach is averaging the normalized expression of all test genes: test value=(ΔCₜ₁+ΔCₜ₂+ . . . +ΔCₜₙ)/n. As will be apparent to skilled artisans, depending on the test genes used, different weight can also be given to different test genes in the present invention. For example, in some embodiments described above, the plurality of test genes comprises at least 2 HRGs, and the combined weight given to the at least 2 HRGs is at least 40% of the total weight given to all of said plurality of test genes. That is, test value=xΔCₜ₁+yΔCₜ₂+ . . . +zΔCₜₙ, wherein ΔCₜ₁ and ΔCₜ₂ represent the gene expression of the 2 HRGs, respectively, and (x+y)/(x+y+ . . . +z) is at least 40%.

It has been determined that, once the invention reported herein is appreciated, the choice of individual HRGs for a test panel can in some embodiments be somewhat arbitrary. In other words, many HRGs have been found to be very good surrogates for each other. One way of assessing whether particular HRGs will serve well in the methods and compositions of the invention is by assessing their correlation with the mean expression of HRGs (e.g., all known HRGs, a specific set of HRGs, etc.). Those HRGs that correlate particularly well with the mean are expected to perform well in assays of the invention, e.g., because these will reduce noise in the assay. Rankings of select HRGs according to their correlation with the mean HRG expression are given in Tables 5, 6, 7, 10, 14, 15, 19, 20, 21, 22, or 23.

Thus, in some embodiments of each of the various aspects of the invention the plurality of test genes comprises the top 2, 3, 4, 5, 6, 7, 8, 9, 10, 11, 12, 13, 14, 15, 20, 25, 30, 35, 40 or more HRGs listed in any of Tables 5, 6, 7, 10, 11, 12, 13, 14, 15, 16, 17, 18, 19, 20, 21, 22, or 23. In some embodiments the plurality of test genes comprises at least some number of HRGs (e.g., at least 3, 4, 5, 6, 7, 8, 9, 10, 15, 20, 25, 30, 35, 40, 45, 50 or more HRGs) and this plurality of HRGs comprises at least 1, 2, 3, 4, 5, 6, 7, 8, 9, 10, 15, 20, or 23 of the following genes: ACTN1, ADM, ANGPTL4, BHLHE40, COL5A2, DDIT4, DUSP1, FOS, LGALS1, LOX, LOXL2, NDRG1, PDGFB, PLAU, PLAUR, SERPINE1, SERPINH1, SLC2A3, STC1, TGFB1, TMEM45A, TNFAIP6, and/or VEGFA. In some embodiments the plurality of test genes comprises at least some number of HRGs (e.g., at least 3, 4, 5, 6, 7, 8, 9, 10, 15, 20, 25, 30, 35, 40, 45, 50 or more HRGs) and this plurality of HRGs comprises at least 1, 2, 3, 4, 5, 6, 7, 8, 9, 10, 15, 20, or 23 of the following genes: ACTN1, ADM, ANGPTL4, COL5A2, DDIT4, DUSP1, ERO1L, FOS, LGALS1, LOX, LOXL2, NDRG1, PDGFB, PGK1, PLAU, PLAUR, SERPINE1, SERPINH1, SLC16A3, SLC2A1, STC1, TMEM45A, and/or TNFAIP6.

In some embodiments the plurality of test genes comprises at least some number of HRGs (e.g., at least 3, 4, 5, 6, 7, 8, 9, 10, 15, 20, 25, 30, 35, 40, 45, 50 or more HRGs) and this plurality of HRGs comprises any one, two, three, four, five, six, seven, eight, nine, ten or 11 or all of gene numbers 1 & 2, 1 to 3, 1 to 4, 1 to 5, 1 to 6, 1 to 7, 1 to 8, 1 to 9, 1 to 10, or 1 to 11 of any of Tables 5, 6, 7, 10, 11, 12, 13, 14, 15, 16, 17, 18, 19, 20, 21, 22, or 23. In some embodiments the plurality of test genes comprises at least some number of HRGs (e.g., at least 3, 4, 5, 6, 7, 8, 9, 10, 15, 20, 25, 30, 35, 40, 45, 50 or more HRGs) and this plurality of HRGs comprises any one, two, three, four, five, six, seven, eight, nine, or ten or all of gene numbers 1 & 2, 1 to 3, 1 to 4, 1 to 5, 1 to 6, 1 to 7, 1 to 8, 1 to 9, or 1 to 10 of any of Tables 5, 6, 7, 10, 11, 12, 13, 14, 15, 16, 17, 18, 19, 20, 21, 22, or 23. In some embodiments the plurality of test genes comprises at least some number of HRGs (e.g., at least 3, 4, 5, 6, 7, 8, 9, 10, 15, 20, 25, 30, 35, 40, 45, 50 or more HRGs) and this plurality of HRGs comprises any one, two, three, four, five, six, seven, eight, or nine or all of gene numbers 2 & 3, 2 to 4, 2 to 5, 2 to 6, 2 to 7, 2 to 8, 2 to 9, or 2 to 10 of any of Tables 5, 6, 7, 10, 11, 12, 13, 14, 15, 16, 17, 18, 19, 20, 21, 22, or 23. In some embodiments the plurality of test genes comprises at least some number of HRGs (e.g., at least 3, 4, 5, 6, 7, 8, 9, 10, 15, 20, 25, 30, 35, 40, 45, 50 or more HRGs) and this plurality of HRGs comprises any one, two, three, four, five, six, seven, or eight or all of gene numbers 3 & 4, 3 to 5, 3 to 6, 3 to 7, 3 to 8, 3 to 9, or 3 to 10 of any of Tables 5, 6, 7, 10, 11, 12, 13, 14, 15, 16, 17, 18, 19, 20, 21, 22, or 23. In some embodiments the plurality of test genes comprises at least some number of HRGs (e.g., at least 3, 4, 5, 6, 7, 8, 9, 10, 15, 20, 25, 30, 35, 40, 45, 50 or more HRGs) and this plurality of HRGs comprises any one, two, three, four, five, six, or seven or all of gene numbers 4 & 5, 4 to 6, 4 to 7, 4 to 8, 4 to 9, or 4 to 10 of any of Tables 5, 6, 7, 10, 11, 12, 13, 14, 15, 16, 17, 18, 19, 20, 21, 22, or 23. In some embodiments the plurality of test genes comprises at least some number of HRGs (e.g., at least 3, 4, 5, 6, 7, 8, 9, 10, 15, 20, 25, 30, 35, 40, 45, 50 or more HRGs) and this plurality of HRGs comprises any one, two, three, four, five, six, seven, eight, nine, 10, 11, 12, 13, 14, or 15 or all of gene numbers 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Tables 5, 6, 7, 10, 11, 12, 13, 14, 15, 16, 17, 18, 19, 20, 21, 22, or 23. In some embodiments the plurality of test genes comprises at least some number of HRGs (e.g., at least 3, 4, 5, 6, 7, 8, 9, 10, 15, 20, 25, 30, 35, 40, 45, 50 or more HRGs) and this plurality of HRGs comprises any one, two, three, four, five, six, seven, eight, nine, 10, 11, 12, 13, 14, or 15 or all of gene numbers 14 & 15, 13 to 15, 12 to 15, 11 to 15, 10 to 15, 9 to 15, 8 to 15, 7 to 15, 6 to 15, 5 to 15, 4 to 15, 3 to 15, 2 to 15, or 1 to 15 of any of Tables 5, 6, 7, 10, 11, 12, 13, 14, 15, 16, 17, 18, 19, 20, 21, 22, or 23.

In some embodiments the plurality of test genes comprises at least some number of HRGs (e.g., at least 3, 4, 5, 6, 7, 8, 9, 10, 15, 20, 25, 30, 35, 40, 45, 50 or more HRGs) and this plurality of HRGs comprises gene numbers 1 & 2; 1 & 2-3; 1 & 3-4; 1 & 4-5; 1 & 5-6; 1 & 6-7; 1 & 7-8; 1 & 8-9; 1 & 9 & 10; 1 & 10 & 11; 1 & 3; 1 & 2-4; 1 & 3-5; 1 & 4-6; 1 & 5-7; 1 & 6-8; 1 & 7-9; 1 & 8-10; 1 & 9 & 11; 1 & 4; 1 & 2-5; 1 & 3-6; 1 & 4-7; 1 & 5-8; 1 & 6-9; 1 & 7-10; 1 & 8-11; 1 & 5; 1 & 2-6; 1 & 3-7; 1 & 4-8; 1 & 5-9; 1 & 6-10; 1 & 7-11; 1 & 6; 1 & 2-7; 1 & 3-8; 1 & 4-9; 1 & 5-10; 1 & 6-11; 1 & 7; 1 & 2-8; 1 & 3-9; 1 & 4-10; 1 & 5-11; 1 & 8; 1 & 2-9; 1 & 3-10; 1 & 4-11; 1 & 9; 1 & 2-10; 1 & 3-11; 1 & 10; 1 & 2-11; 1 & 11; 2 & 3; 2 & 3-4; 2 & 4-5; 2 & 5-6; 2 & 6-7; 2 & 7-8; 2 & 8-9; 2 & 9 & 10; 2 & 10 & 11; 2 & 4; 2 & 3-5; 2 & 4-6; 2 & 5-7; 2 & 6-8; 2& 7-9; 2 & 8-10; 2 & 9 & 11; 2 & 5; 2 & 3-6; 2 & 4-7; 2 & 5-8; 2 & 6-9; 2 & 7-10; 2 & 8-11; 2 & 6; 2 & 3-7; 2 & 4-8; 2 & 5-9; 2 & 6-10; 2 & 7-11; 2 & 7; 2 & 3-8; 2 & 4-9; 2 & 5-10; 2 & 6-11; 2 & 8; 2 & 3-9; 2 & 4-10; 2 & 5-11; 2 & 9; 2 & 3-10; 2 & 4-11; 2 & 10; 2 & 3-11; 2 & 11; 3 & 4; 3 & 4-5; 3 & 5-6; 3 & 6-7; 3 & 7-8; 3 & 8-9; 3 & 9 & 10; 3 & 10 & 11; 3 & 5; 3 & 4-6; 3 & 5-7; 3 & 6-8; 3& 7-9; 3 & 8-10; 3 & 9 & 11; 3 & 6; 3 & 4-7; 3 & 5-8; 3 & 6-9; 3 & 7-10; 3 & 8-11; 3 & 7; 3 & 4-8; 3 & 5-9; 3 & 6-10; 3 & 7-11; 3 & 8; 3 & 4-9; 3 & 5-10; 3 & 6-11; 3 & 9; 3 & 4-10; 3 & 5-11; 3 & 10; 3 & 4-11; 3 & 11; 4 & 5; 4 & 5-6; 4 & 6-7; 4 & 7-8; 4 & 8-9; 4 & 9 & 10; 4 & 10-11; 4 & 6; 4 & 5-7; 4 & 6-8; 4 & 7-9; 4 & 8-10; 4 & 9-11; 4 & 7; 4 & 5-8; 4 & 6-9; 4 & 7-10; 4 & 8-11; 4 & 8; 4 & 5-9; 4 & 6-10; 4 & 7-11; 4 & 9; 4 & 5-10; 4 & 6-11; 4 & 10; 4 & 5-11; 4 & 11; 5 & 6; 5 & 6-7; 5 & 7-8; 5 & 8-9; 5 & 9 & 10; 5 & 10-11; 5 & 7; 5 & 6-8; 5 & 7-9; 5 & 8-10; 5 & 9-11; 5 & 8; 5 & 6-9; 5 & 7-10; 5 & 8-11; 5 & 9; 5 & 6-10; 5 & 7-11; 5 & 10; 5 & 6-11; 5 & 11; 6 & 7; 6 & 7-8; 6 & 8-9; 6 & 9 & 10; 6 & 10-11; 6 & 8; 6 & 7-9; 6 & 8-10; 6 & 9-11; 6 & 9; 6 & 7-10; 6 & 8-11; 6 & 10; 6 & 7-11; 6 & 11; 7 & 8; 7 & 8-9; 7 & 9 & 10; 7 & 10-11; 7 & 9; 7 & 8-10; 7 & 9-11; 7 & 10; 7 & 8-11; 7 & 11; 8 & 9; 8 & 9-10; 8 & 10-11; 8 & 10; 8 & 9-11; 8 & 11; 9 & 10; 9 & 10-11; or gene numbers 9 & 11 of any of Tables 5, 6, 7, 10, 11, 12, 13, 14, 15, 16, 17, 18, 19, 20, 21, 22, or 23.

In some embodiments the plurality of test genes comprises at least some number of HRGs (e.g., at least 3, 4, 5, 6, 7, 8, 9, 10, 15, 20, 25, 30, 35, 40, 45, 50 or more HRGs; including at least 3, 4, 5, 6, 7, 8, 9, 10, 15, 20, 25, 30, 35, 40, 45, 50 or more HRGs from any of Tables 1, 2, 5, 6, 7, 10, 19, 20, or 21) and this plurality of HRGs does not include one or more of the following genes: ADM, ALDOA, ALDOA, ANGPTL4, BHLHB2, C3orf28, CA9, CA9, DDIT4, DUSP1, EGFR, FOS, GJA, GJA1, GNB2L1, HIG2, IGF2, IGFBP3, IGFBP5, INHA, INHBB, LDHA, LOX, LOXL2, MIF, MXI1, NDRG1, P4HA1, PDGFB, PFKFB3, PGK1, PLOD2, RNASE4, SERPINE1, SLC16A3, SLC2A1, SOX9, SSR4, STC1, TEFL TMEM45A, TPI1, VEGFA, ZFP36L2, or ZNF395.

In some embodiments the plurality of test genes comprises at least some number of HRGs (e.g., at least 3, 4, 5, 6, 7, 8, 9, 10, 15, 20, 25, 30, 35, 40, 45, 50 or more HRGs; including at least 3, 4, 5, 6, 7, 8, 9, 10, 15, 20, 25, 30, 35, 40, 45, 50 or more HRGs from any of Tables 1, 2, 5, 6, 7, 10, 19, 20 or 21) and this plurality of HRGs does not include SLC2A1, VEGFA, PGK1, LDHA, TPI1, CA9, ALDOA, P4HA1, ANGPTL4, and HIG2; or ANGPTL4, BHLHB2, C3orf28, DDIT4, PFKFB3, RNASE4, SERPINE1, SLC16A3, VEGFA, and ZNF395; or SOX9; or DUSP1, FOS, IGFBP3, IGFBP5, and LOX; or SERPINE1, ADM, INHA, STC1, SLC2A1, and ALDOA; or INHA, SLC2A1, and STC1; or MIF; or ZFP36L2, DUSP1, EGFR, FOS, IGF2, INHA, MXI1, and PDGFB; or CA9; or TEF1, SSR4, INHBB, TMEM45A, PGK1, SOX9, FOS, DUSP1, TMEM45A, and GJA; or GNB2L1; or LOX, FOS, IGFBP3, and IGFBP5; or NDRG1; or FOS, LOXL2, PLOD2, and ADM; or SERPINE1 and GJA1; or SERPINE1, SOX9, LOXL2, and TMEM45A; or IGFBP3, FOS, SERPINE1, SLC2A1, PGK1, and MIF; or EGFR.

### II. Cancer Prognosis

It has been surprisingly discovered that in selected cancers (e.g., lung cancer and colon cancer) the expression of HRGs in tumor cells can accurately predict the degree of aggression of the cancer and risk of recurrence after treatment (e.g., surgical removal of cancer tissue, chemotherapy, radiation therapy, etc.). Thus, the above-described method of determining HRG expression can be applied in the prognosis and treatment of these cancers. For this purpose, the description above about the method of determining HRG expression is incorporated herein.

Generally, a method is further provided for prognosing cancer (e.g., selected from lung cancer and colon cancer), which comprises determining in a tumor sample from a cancer patient (e.g., a patient diagnosed with lung cancer or colon cancer), the expression of at least 2, 4, 5, 6, 7 or at least 8, 9, 10 or 12 HRGs, wherein high expression (or increased expression or overexpression) of the 2, 4, 5, 6, 7 or at least 8, 9, 10 or 12 HRGs indicates a poor prognosis or an increased likelihood of progression or recurrence of cancer in the patient. The expression can be determined in accordance with the method described above. In some embodiments, the method comprises at least one of the following steps: (a) correlating high expression (or increased expression or overexpression) of the 2, 4, 5, 6, 7 or at least 8, 9, 10 or 12 HRGs to a poor prognosis or an increased likelihood of progression or recurrence of cancer in the patient; (b) concluding that the patient has a poor prognosis or an increased likelihood of progression or recurrence of cancer based at least in part on high expression (or increased expression or overexpression) of the 2, 4, 5, 6, 7 or at least 8, 9, 10 or 12 HRGs; or (c) communicating that the patient has a poor prognosis or an increased likelihood of progression or recurrence of cancer based at least in part on high expression (or increased expression or overexpression) of the 2, 4, 5, 6, 7 or at least 8, 9, 10 or 12 HRGs.

In each embodiment described in this document involving correlating a particular assay or analysis output (e.g., high HRG expression, test value incorporating HRG expression greater than some reference value, etc.) to some likelihood (e.g., increased, not increased, decreased, etc.) of some clinical event or outcome (e.g., recurrence, progression, cancer-specific death, etc.), such correlating may comprise assigning a risk or likelihood of the clinical event or outcome occurring based at least in part on the particular assay or analysis output. In some embodiments, such risk is a percentage probability of the event or outcome occurring. In some embodiments, the patient is assigned to a risk group (e.g., low risk, intermediate risk, high risk, etc.). In some embodiments “low risk” is any percentage probability below 5%, 10%, 15%, 20%, 25%, 30%, 35%, 40%, 45%, or 50%. In some embodiments “intermediate risk” is any percentage probability above 5%, 10%, 15%, 20%, 25%, 30%, 35%, 40%, 45%, or 50% and below 15%, 20%, 25%, 30%, 35%, 40%, 45%, 50%, 55%, 60%, 65%, 70%, or 75%. In some embodiments “high risk” is any percentage probability above 25%, 30%, 35%, 40%, 45%, 50%, 55%, 60%, 65%, 70%, 75%, 80%, 85%, 90%, 95%, or 99%.

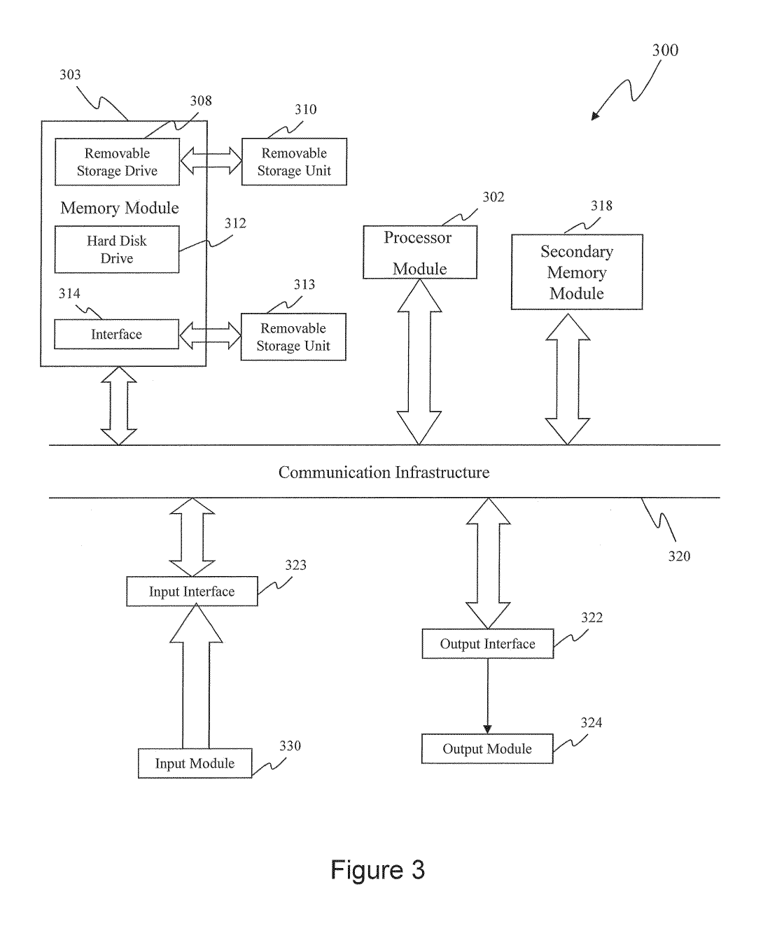

As used herein, “communicating” a particular piece of information means to make such information known to another person or transfer such information to a thing (e.g., a computer). In some methods of the invention, a patient's prognosis or risk of recurrence is communicated. In some embodiments, the information used to arrive at such a prognosis or risk prediction (e.g., expression levels of a panel of biomarkers comprising a plurality of HRGs, clinical or pathologic factors, etc.) is communicated. This communication may be auditory (e.g., verbal), visual (e.g., written), electronic (e.g., data transferred from one computer system to another), etc. In some embodiments, communicating a cancer classification comprises generating a report that communicates the cancer classification. In some embodiments the report is a paper report, an auditory report, or an electronic record. In some embodiments the report is displayed and/or stored on a computing device (e.g., handheld device, desktop computer, smart device, website, etc.). In some embodiments the cancer classification is communicated to a physician (e.g., a report communicating the classification is provided to the physician). In some embodiments the cancer classification is communicated to a patient (e.g., a report communicating the classification is provided to the patient). Communicating a cancer classification can also be accomplished by transferring information (e.g., data) embodying the classification to a server computer and allowing an intermediary or end-user to access such information (e.g., by viewing the information as displayed from the server, by downloading the information in the form of one or more files transferred from the server to the intermediary or end-user's device, etc.).

Wherever an embodiment of the invention comprises concluding some fact (e.g., a patient's prognosis or a patient's likelihood of recurrence), this may include a computer program concluding such fact, typically after performing some algorithm that incorporates information on the status of HRGs in a patient sample (e.g., as shown in FIG. 3).

In one embodiment, the prognosis method comprises (1) determining in a sample the expression of a panel of biomarkers including at least 4, 5, 6, or at least 8 HRGs; and (2) providing a test value by (a) weighting the determined expression of each of a plurality of test genes selected from the panel of biomarkers with a predefined coefficient, and (b) combining the weighted expression to provide the test value, wherein the combined weight given to said at least 4 or 5 or 6 HRGs is at least 40% (or 50%, 60%, 70%, 80%, 90%, 95% or 100%) of the total weight given to the expression of all of said plurality of test genes, and wherein high expression (or increased expression or overexpression) of the plurality of test genes indicates the patient has a poor prognosis or an increased likelihood that the patient's cancer will progress aggressively. In some embodiments, the method comprises at least one of the following steps: (a) correlating high expression (or increased expression or overexpression) of the plurality of test genes to a poor prognosis or an increased likelihood that the patient's cancer will progress aggressively; (b) concluding that the patient has a poor prognosis or an increased likelihood of progression or recurrence of cancer based at least in part on high expression (or increased expression or overexpression) of the plurality of test genes; or (c) communicating that the patient has a poor prognosis or an increased likelihood that the patient's cancer will progress aggressively based at least in part on high expression (or increased expression or overexpression) of the plurality of test genes.

In some embodiments at least 20%, 50%, 75%, or 90% of said plurality of test genes are HRGs.

In some embodiments, the prognosis method further includes a step of comparing the test value provided in step (2) above to one or more reference values, and correlating the test value to the prognosis of cancer. Optionally poor prognosis of the cancer is indicated if the test value is greater than the reference value.

In some embodiments, said plurality of test genes includes at least 2 HRGs which constitute at least 50% or at least 60% of said plurality of test genes. In some embodiments, said plurality of test genes includes at least 4 HRGs which constitute at least 20% or 30% or 50% or 60% of said plurality of test genes.

In some embodiments, said plurality of test genes comprises at least 2 HRGs, and the combined weight given to said at least 2 HRGs is at least 40% (or 50%, 60%, 70%, 80%, 90%, 95% or 100%) of the total weight given to the expression of all of said plurality of test genes. In some embodiments, said plurality of test genes comprises at least 4 or 5 or 6 HRGs, and the combined weight given to said at least 4 or 5 or 6 HRGs is at least (or 50%, 60%, 70%, 80%, 90%, 95% or 100%) of the total weight given to the expression of all of said plurality of test genes.

In some embodiments, said plurality of test genes comprises one or more HRGs constituting from 1% to about 95% of said plurality of test genes, and the combined weight given to said one or more HRGs is (or 50%, 60%, 70%, 80%, 90%, 95% or 100%) of the total weight given to the expression of all of said plurality of test genes. Preferably, said plurality of test genes includes at least 2, preferably 4, more preferably at least 5 HRGs, and most preferably at least 6 HRGs.

In some embodiments, said plurality of test genes includes the HRGs INHBA and FAP. In some embodiments, said panel of biomarkers in the method described above comprises INHBA and FAP, and said plurality of test genes includes INHBA and FAP, and optionally the weighting of the expression of the test genes is according to that in O'Connell et al., J. CLIN. ONCOL. (2010) 28:3937-3944, which is incorporated herein by reference.

In the various embodiments described above, preferably the weight coefficient given to each HRG in said plurality of test genes is greater than 1/N where N is the total number of test genes in the plurality of test genes.

In some embodiments, the prognosis method includes (1) obtaining a tumor sample from a patient identified as having lung cancer or colon cancer; (2) determining the expression of a panel of biomarkers in the tumor sample including at least 2, 4, 6, 8 or 10 HRGs; and (3) providing a test value by (a) weighting the determined expression of each of a plurality of test genes selected from the panel of biomarkers with a predefined coefficient, and (b) combining the weighted expression to provide said test value, wherein the combined weight given to said at least 4 or 5 or 6 HRGs is at least 40% (or 50%, 60%, 70%, 80%, 90%, 95% or 100%) of the total weight given to the expression of all of said plurality of test genes, and wherein high expression (or increased expression or overexpression) of the plurality of test genes indicates a poor prognosis or an increased likelihood of cancer recurrence. In some embodiments, the method comprises at least one of the following steps: (a) correlating high expression (or increased expression or overexpression) of the plurality of test genes to a poor prognosis or an increased likelihood of cancer recurrence; (b) concluding that the patient has a poor prognosis or an increased likelihood of cancer recurrence based at least in part on high expression (or increased expression or overexpression) of the plurality of test genes; or (c) communicating that the patient has a poor prognosis or an increased likelihood of cancer recurrence based at least in part on high expression (or increased expression or overexpression) of the plurality of test genes. In some embodiments at least 20%, 50%, 75%, or 90% of said plurality of test genes are HRGs.

Some embodiments provide a method for prognosing cancer comprising: (1) obtaining expression level data, from a sample (e.g., tumor sample) from a patient identified as having lung cancer or colon cancer, for a panel of biomarkers including at least 2, 4, 6, 8 or 10 HRGs; and (2) providing a test value by (a) weighting the determined expression of each of a plurality of test genes selected from said panel of biomarkers with a predefined coefficient, and (b) combining the weighted expression to provide said test value, wherein the combined weight given to said at least 4 or 5 or 6 HRGs is at least 40% (or 50%, 60%, 70%, 80%, 90%, 95% or 100%) of the total weight given to the expression of all of said plurality of test genes. In some embodiments at least 20%, 50%, 75%, or 90% of said plurality of test genes are HRGs.

A related aspect of the invention provides a method of classifying cancer comprising determining the status of a panel of biomarkers comprising at least two HRGs, in tissue or cell sample, particularly a tumor sample, from a patient, wherein an abnormal status indicates a negative cancer classification. The methods of this aspect may comprise at least one of the following steps: (a) correlating abnormal status of the HRGs to a negative cancer classification; (b) concluding that the patient has a negative cancer classification based at least in part on abnormal status of the HRGs; or (c) communicating that the patient has a negative cancer classification based at least in part on abnormal status of the HRGs. As used herein, “determining the status” of a biomarker refers to determining the presence, absence, or extent/level of some physical, chemical, or genetic characteristic of the biomarker. In cases where the biomarker is a gene, such characteristics include, but are not limited to, expression levels, activity levels, mutations, copy number, methylation status, etc. Unless the text or context indicates otherwise, any reference herein to determining the status of a gene may include either determining the expression level of the mRNA encoded by the gene (or a cDNA reverse transcribed therefrom), determining the expression level of the protein encoded by the gene, or both.

In the context of HRGs as used to determine risk of cancer recurrence or progression or determine the need for aggressive treatment, particularly useful characteristics include expression levels (e.g., mRNA or protein levels) and activity levels. Characteristics may be assayed directly (e.g., by assaying a HRG's expression level) or determined indirectly (e.g., assaying the level of a gene or genes whose expression level is correlated to the expression level of the HRG). Thus some embodiments of the invention provide a method of classifying cancer comprising determining the expression level, particularly mRNA (alternatively cDNA) level, of a panel of genes comprising at least two HRGs, in a tumor sample, wherein high expression (or increased expression or overexpression) indicates the patient has (a) a negative cancer classification, (b) an increased risk of cancer recurrence or progression, or (c) a need for aggressive treatment. In some embodiments, the method comprises at least one of the following steps: (a) correlating high expression (or increased expression or overexpression) of the panel of genes to a negative cancer classification, an increased risk of cancer recurrence or progression, or a need for aggressive treatment; (b) concluding that the patient has a negative cancer classification, an increased risk of cancer recurrence or progression, or a need for aggressive treatment based at least in part on high expression (or increased expression or overexpression) of the panel of genes; or (c) communicating that the patient has a negative cancer classification, an increased risk of cancer recurrence or progression, or a need for aggressive treatment based at least in part on high expression (or increased expression or overexpression) of the panel of genes. In some embodiments, as shown in Example 4, below, increased expression of HRGs (e.g., a panel of plurality of HRGs in a plurality of test genes) indicates adjuvant chemotherapy is not appropriate (or there is a lower likelihood of response) for the patient. Thus in some embodiments, the method further comprises correlating increased HRG expression with a lower likelihood of response to adjuvant chemotherapy (e.g., in colorectal cancer patients).

“Abnormal status” means a marker's status in a particular sample differs from the status generally found in average samples (e.g., healthy samples or average diseased samples). Examples include mutated, elevated (or increased), decreased, present, absent, negative, positive, etc. In this context, a “negative status” generally means the characteristic is absent or undetectable. For example, LGALS1 status is negative if LGALS1 nucleic acid and/or protein is absent or undetectable in a sample. However, negative LGALS1 status also includes a mutation or copy number reduction in LGALS1 LGALS1.

Generally the invention provides methods where abnormal HRG expression indicates a negative cancer classification. “Abnormal expression” means a gene's expression level in a particular sample differs from the level generally found in average samples (e.g., healthy samples, average diseased samples, etc.). Examples of “abnormal expression” include elevated, decreased, present, absent, etc. An “elevated expression” or “increased expression” means that the level of one or more of the above expression products (e.g., mRNA) is higher than normal levels. Generally this means an increase in the level (e.g., mRNA level) as compared to an index value. Conversely a “low expression” or “decreased expression” means that the level of one or more of the above expression products (e.g., mRNA) is lower than normal levels. Generally this means a decrease in the level (e.g., mRNA level) as compared to an index value. In this context, “low expression” can include absent or undetectable expression.

In some embodiments, the test value representing the expression (e.g., overall expression) of the plurality of test genes is compared to one or more reference values (or index values), and optionally correlated to a risk of cancer progression or risk of cancer recurrence. Optionally an increased likelihood of poor prognosis is indicated if the test value is greater than the reference value. Thus, a “test value” determined to reflect the expression of a plurality of genes will generally be compared with a reference or index value.

Those skilled in the art are familiar with various ways of deriving and using index values. For example, the index value may represent the levels of a biomarker found in a normal sample obtained from the patient of interest, in which case a level in the tumor sample significantly higher (e.g., 1.5-fold, 2-fold, 3-fold, 4-fold, 5-fold, 10-fold, 20-fold, 30-fold, 40-fold, 50-fold, 100-fold or more higher) than this index value would indicate, e.g., a poor prognosis or increased likelihood of cancer recurrence or a need for aggressive treatment.

Often the leve of a biomarker will be considered “increased” or “decreased” only if it differs significantly from the index value. Thus in some embodiments levels are deemed “increased” over the index value only if they are at least some amount or fold change (including some number of standard deviations) higher than the index value. Similarly, in some embodiments levels are deemed “decreased” below the index value only if they are at least some amount or fold change lower than the index value. For example, in some embodiments an “increased” or “decreased” level means the level in the sample is at least 25%, 30%, 35%, 40%, 45%, 50%, 55%, 60%, 65%, 70%, 75%, 80%, 85%, 90%, 95% or more higher or lower than the index value. In some embodiments an “increased” or “decreased” level means the level in the sample is at least 1.5, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900, or 1000 or more fold higher or lower than the index value. In some embodiments an “increased” or “decreased” level means the level in the sample is at least 1, 2, 3, 4, 5, 6, 7, 8, 9, 10 or more standard deviations higher or lower than the index value.

Alternatively, the index value may represent the average level for a set of individuals from a diverse cancer population or a subset of the population. For example, one may determine the average level of a biomarker or biomarker panel in a random sampling of patients with cancer (e.g., lung or colorectal cancer). This average level may be termed the “threshold index value,” with patients having levels (e.g., HRG expression levels) higher than this value expected to have a poorer prognosis than those having levels lower than this value. Alternatively the “threshold index value” may be a value some statistically significant amount higher than this average level. In some embodiments the threshold index value is 1.5-fold, 2-fold, 3-fold, 4-fold, 5-fold, 10-fold, 20-fold, 30-fold, 40-fold, 50-fold, 100-fold or more higher than the average level. In some embodiments the threshold index value is 1, 2, 3, 4, 5, 6, 7, 8, 9, 10 or more standard deviations higher than the average level. In some embodiments the reference population is divided into groups (e.g., terciles, quartiles, quintiles), with each group assigned one or more index values (e.g., the average level across members of each group, levels representing the boundaries of each group, etc.).

Alternatively the index value may represent the average level of a particular biomarker in a plurality of training patients (e.g., healthy controls, lung or colon cancer patients) with similar clinical features (e.g., similar outcomes whose clinical and follow-up data are available and sufficient to define and categorize the patients by disease outcome, e.g., recurrence or prognosis). See, e.g., Examples, infra. For example, a “good prognosis index value” can be generated from a plurality of training cancer patients characterized as having “good outcome”, e.g., those who have not had cancer recurrence five years (or ten years or more) after initial treatment, or who have not had progression in their cancer five years (or ten years or more) after initial diagnosis. A “poor prognosis index value” can be generated from a plurality of training cancer patients defined as having “poor outcome”, e.g., those who have had cancer recurrence within five years (or ten years, etc.) after initial treatment, or who have had progression in their cancer within five years (or ten years, etc.) after initial diagnosis. Thus, a good prognosis index value of a particular biomarker may represent the average level of the particular biomarker in patients having a “good outcome,” whereas a poor prognosis index value of a particular biomarker represents the average level of the particular biomarker in patients having a “poor outcome.”

Thus, when the determined level of a relevant biomarker is closer to the good prognosis index value of the biomarker than to the poor prognosis index value of the biomarker, then it can be concluded that the patient is more likely to have a good prognosis, e.g., a low (or no increased) likelihood of cancer recurrence. On the other hand, if the determined level of a relevant biomarker is closer to the poor prognosis index value of the biomarker than to the good prognosis index value of the biomarker, then it can be concluded that the patient is more likely to have a poor prognosis, e.g., an increased likelihood of cancer recurrence.

Alternatively index values may be determined thusly: In order to assign patients to risk groups (e.g., high likelihood of having cancer, high likelihood of recurrence/progression), a threshold value will be set for the HRG mean. The optimal threshold value is selected based on the receiver operating characteristic (ROC) curve, which plots sensitivity vs (1—specificity). For each increment of the HRG mean, the sensitivity and specificity of the test is calculated using that value as a threshold. The actual threshold will be the value that optimizes these metrics according to the artisan's requirements (e.g., what degree of sensitivity or specificity is desired, etc.).

Panels of HRGs (e.g., at least 2, 3, 4, 5, 6, 7, 8, 9, 10, 12, 14, 16, 18, 20, 25, 30, 35, 40, 45, 50, 60, 70, 80, 90, or 100 or more HRGs) can predict prognosis of cancer (Examples below). Those skilled in the art are familiar with various ways of determining the expression of a panel (i.e., a plurality) of genes, including the techniques discussed above for determining test values for gene panels. Sometimes herein this is called determining the “overall expression” of a panel or plurality of genes. One may determine the expression of a panel of genes by determining the average expression level (normalized or absolute) of all panel genes in a sample obtained from a particular patient (either throughout the sample or in a subset of cells from the sample or in a single cell). Increased expression in this context will mean the average expression is higher than the average expression level of these genes in normal patients (or higher than some index value that has been determined to represent the average expression level in a reference population such as healthy patients or patients with a particular cancer). Alternatively, one may determine the expression of a panel of genes by determining the average expression level (normalized or absolute) of at least a certain number (e.g., 1, 2, 3, 4, 5, 6, 7, 8, 9, 10, 15, 20, 25, 30 or more) or at least a certain proportion (e.g., 10%, 20%, 30%, 40%, 50%, 60%, 70%, 80%, 90%, 95%, 99%, 100%) of the genes in the panel. Alternatively, one may determine the expression of a panel of genes by determining the absolute copy number of the mRNA (or protein) of all the genes in the panel and either total or average these across the genes.

As used herein, “classifying a cancer” and “cancer classification” refer to determining one or more clinically-relevant features of a cancer and/or determining a particular prognosis of a patient having said cancer. Thus “classifying a cancer” includes, but is not limited to: (i) evaluating metastatic potential, potential to metastasize to specific organs, risk of recurrence, and/or course of the tumor; (ii) evaluating tumor stage; (iii) determining patient prognosis in the absence of treatment of the cancer; (iv) determining prognosis of patient response (e.g., tumor shrinkage or progression-free survival) to treatment (e.g., chemotherapy, radiation therapy, surgery to excise tumor, etc.); (v) diagnosis of actual patient response to current and/or past treatment; (vi) determining a preferred course of treatment for the patient; (vii) prognosis for patient relapse after treatment (either treatment in general or some particular treatment); (viii) prognosis of patient life expectancy (e.g., prognosis for overall survival), etc.

Thus, a “negative classification” means an unfavorable clinical feature of the cancer (e.g., a poor prognosis). Examples include (i) an increased metastatic potential, potential to metastasize to specific organs, and/or risk of recurrence; (ii) an advanced tumor stage; (iii) a poor patient prognosis in the absence of treatment of the cancer; (iv) a poor prognosis of patient response (e.g., tumor shrinkage or progression-free survival) to a particular treatment (e.g., chemotherapy, radiation therapy, surgery to excise tumor, etc.); (v) a poor prognosis for patient relapse after treatment (either treatment in general or some particular treatment); (vi) a poor prognosis of patient life expectancy (e.g., prognosis for overall survival), etc. In some embodiments a recurrence-associated clinical parameter (or a high nomogram score) and increased expression of a HRG indicate (or are correlated to) a negative classification in cancer (e.g., increased likelihood of recurrence or progression).

In some embodiments a combined score (e.g., prognosis score) can be derived from HRG status together with one or more clinical variables (which themselves can be combined into a component score, e.g., clinical variable score). These clinical variables can include age, gender, smoking status (particularly in the case of lung cancer patients), pathological stage, tumor size, adjuvant treatment, pleural invasion, cytology, serum CEA, serum CA19-9, and grade. In some embodiments the combined score is calculated according to the following equation:

Combined Score=A*(HRG Score)+B*(Clinical Variable Score)+C*(Other Components)

The “HRG Score” can be any of the test values described in this document that incorporate HRG status (e.g., test value calculated from expression of a plurality of test genes where HRGs are weighted to contribute at least some minimum weight to the test value). In some embodiments HRG Score can be the unweighted mean of C_{T} values for expression of the HRGs being analyzed, optionally normalized by the unweighted mean of the control genes so that higher values indicate higher expression (in some embodiments one unit is equivalent to a two-fold change in expression). In some embodiments the HRG Score ranges from −8 to 8 or from −1.6 to 3.7.

The “Clinical Variable Score” can be any score derived from one or more clinical variables, wherein the clinical variables are assigned some numerical value based on the patient's status and then combined to yield a numerical score (which is then weighted by the factor B in the Combined Score). In some embodiments, the Clinical Variable Score incorporates the following clinical variables, or any combination thereof, as shown:

TABLE B

| | Possible Observed | |
|---|---|---|
| | Clinical Status/ | Corresponding Assigned Values |
| Clinical Variable | Values | for Clinical Variable Score |
| Age | Age in Years | Continuous (number of years) |
| Gender | Male or Female | 0 or 1 |
| Tumor Grade | 1, 2, or 3 | 1, 2, or 4 |
| Tumor Location | Left or Right | 0 or 1 |
| T stage | T1, T2, T3, or T4a | 0, 1, 2, or 3 |
| N stage | N0 or N1 | 0 or 1 |
| Number of Nodes | Number of Nodes | Continuous (number of nodes) |
| Examined | | or binary (<12 = 0, 12 = 1) |
| Adjuvant | Yes or No | 0 or 1 |
| Treatment | | |

In some embodiments the Combined Score consists of the HRG Score combined with the Clinical Variable Score; i.e., in such embodiments C=0 because there are no Other Components. Otherwise, “Other Components” can be any additional clinical or other factors that may be combined with HRG Score and Clinical Variable Score to yield a Combined Score that classifies the cancer.

In some embodiments A=1, B=1 and, if not zero, then C=1. In some embodiments A is between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20; B is between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20; and C is 0 or between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20. In some embodiments, A, B, and/or C is within rounding of any of these values (e.g., A is between 0.45 and 0.54, etc.).

As discussed above, test values calculated at least in part from high HRG expression levels in a patient sample have been shown to often mean the patient has an increased likelihood of recurrence after treatment (e.g., the cancer cells not killed or removed by the treatment will quickly grow back); the patient has an increased likelihood of cancer progression for more rapid progression (e.g., the rapidly proliferating cells will cause any tumor to grow quickly, gain in virulence, and/or metastasize); or the patient may require a relatively more aggressive treatment. Thus, in some embodiments the invention provides a method of classifying cancer comprising determining the expression of a panel of genes comprising a plurality of HRGs, wherein an abnormal expression indicates an increased likelihood of recurrence or progression. As discussed above, in some embodiments the expression to be determined is gene expression levels (while in others it is protein expression). Thus in some embodiments the invention provides a method of determining the prognosis of a patient's cancer comprising determining the expression level of a panel of genes comprising a plurality of HRGs, wherein high expression (or increased expression or overexpression) indicates an increased likelihood of recurrence or progression of the cancer. In some embodiments, the method comprises at least one of the following steps: (a) correlating abnormal expression (e.g., high expression (or increased expression or overexpression)) of the panel of genes to an increased likelihood of recurrence or progression; (b) concluding that the patient has an increased likelihood of recurrence or progression based at least in part on abnormal expression (e.g., high expression (or increased expression or overexpression)) of the panel of genes; or (c) communicating that the patient has an increased likelihood of recurrence or progression based at least in part on abnormal expression (e.g., high expression (or increased expression or overexpression)) of the panel of genes.

“Recurrence” and “progression” are terms well-known in the art and are used herein according to their known meanings. As an example, the meaning of “progression” may be cancer-type dependent, with progression in lung cancer meaning something different from progression in prostate cancer. However, within each cancer-type and subtype “progression” is clearly understood to those skilled in the art. As used herein, a patient has an “increased likelihood” of some clinical feature or outcome (e.g., recurrence or progression) if the probability of the patient having the feature or outcome exceeds some reference probability or value. The reference probability may be the probability of the feature or outcome across the general relevant patient population. For example, if the probability of recurrence in the general prostate cancer population is X % and a particular patient has been determined by the methods of the present invention to have a probability of recurrence of Y %, and if Y>X, then the patient has an “increased likelihood” of recurrence. Alternatively, as discussed above, a threshold or reference value may be determined and a particular patient's probability of recurrence may be compared to that threshold or reference. Because predicting recurrence and predicting progression are prognostic endeavors, “predicting prognosis” will often be used herein to refer to either or both. In these cases, a “poor prognosis” will generally refer to an increased likelihood of recurrence, progression, or both.

As shown in Example 3, individual HRGs can predict prognosis quite well. Thus the invention provides methods of predicting prognosis comprising determining the expression of at least one HRG listed in Tables 1, 2, 3, 5, 6, 7, or 10.

The Examples below show that a panel of HRGs can accurately predict prognosis. Thus, as discussed in detail above, in some embodiments the methods of the invention comprise determining the status of a panel (i.e., a plurality) of test genes comprising a plurality of HRGs (e.g., to provide a test value representing the average expression of the test genes). For example, increased expression in a panel of test genes may refer to the average expression level of all panel genes in a particular patient being higher than the average expression level of these genes in normal patients (or higher than some index value that has been determined to represent the normal average expression level). Alternatively, increased expression in a panel of test genes may refer to increased expression in at least a certain number (e.g., 1, 2, 3, 4, 5, 6, 7, 8, 9, 10, 15, 20, 25, 30 or more) or at least a certain proportion (e.g., 10%, 20%, 30%, 40%, 50%, 60%, 70%, 80%, 90%, 95%, 99%, 100%) of the genes in the panel as compared to the average normal expression level.

In some embodiments the test panel (which may itself be a sub-panel analyzed informatically) comprises at least 3, 4, 5, 6, 7, 8, 9, 10, 15, 20, 25, 30, 35, 40, 45, 50, 60, 70, 80, 90, 100, 200, or more HRGs. In some embodiments the test panel comprises at least 10, 15, 20, or more HRGs. In some embodiments the test panel comprises between 5 and 100 HRGs, between 7 and 40 HRGs, between 5 and 25 HRGs, between 10 and 20 HRGs, or between 10 and 15 HRGs. In some embodiments HRGs comprise at least a certain proportion of the test panel used to provide a test value. Thus in some embodiments the test panel comprises at least 25%, 30%, 40%, 50%, 60%, 70%, 75%, 80%, 85%, 90%, 95%, 96%, 97%, 98%, or 99% HRGs. In some embodiments the test panel comprises at least 10, 15, 20, 25, 30, 35, 40, 45, 50, 70, 80, 90, 100, 200, or more HRGs, and such HRGs constitute at least 50%, 60%, 70%, preferably at least 75%, 80%, 85%, more preferably at least 90%, 95%, 96%, 97%, 98%, or 99% or more of the total number of genes in the test panel. In some embodiments the HRGs are chosen from the group consisting of the genes in any of Tables 1, 2, 3, 5, 6, 7, 10, 11, 12, 13, 14, or 15. In some embodiments the test panel comprises at least 2, 3, 4, 5, 6, 7, 8, 9, 10, 11, 12, 13, 14, 15, 20, 25, 30, or more (or all) of the genes in Tables 1, 2, 3, 5, 6, 7, 10, 11, 12, 13, 14, or 15. In some embodiments the invention provides a method of predicting prognosis comprising determining (e.g., in a sample) the status of the genes in Tables 1, 2, 3, 5, 6, 7, 10, 11, 12, 13, 14, or 15, wherein abnormal status (e.g., increased expression) indicates a poor prognosis. In some embodiments, the method comprises at least one of the following steps: (a) correlating abnormal status (e.g., increased expression) of the genes in Tables 1, 2, 3, 5, 6, 7, 10, 11, 12, 13, 14, or 15 to a poor prognosis; (b) concluding that the patient has a poor prognosis based at least in part on abnormal status (e.g., increased expression) of the genes in Tables 1, 2, 3, 5, 6, 7, 10, 11, 12, 13, 14, or 15; or (c) communicating that the patient has a poor prognosis based at least in part on abnormal status (e.g., increased expression) of the genes in Tables 1, 2, 3, 5, 6, 7, 10, 11, 12, 13, 14, or 15.

In some of these embodiments elevated expression indicates an increased likelihood of recurrence or progression. Thus in some embodiments the invention provides a method of predicting risk of cancer recurrence or progression in a patient comprising determining the status of a panel of biomarkers, wherein the panel comprises between about 10 and about 15 HRGs, wherein the combined weight given to said between about 10 and about 15 HRGs is at least 40% (or 50%, 60%, 70%, 80%, 90%, 95% or 100%) of the total weight given to the expression of all of said plurality of test genes, and an elevated status for the HRGs indicates an increased likelihood or recurrence or progression. In some embodiments, the method comprises at least one of the following steps: (a) correlating elevated status (e.g., increased expression) of the panel of biomarkers to an increased likelihood of recurrence or progression; (b) concluding that the patient has an increased likelihood of recurrence or progression based at least in part on elevated status (e.g., increased expression) of the panel of biomarkers; or (c) communicating that the patient has an increased likelihood of recurrence or progression based at least in part on elevated status (e.g., increased expression) of the panel of biomarkers.

It has been determined that, once the hypoxia phenomenon reported herein is appreciated, the choice of individual HRGs for a test panel can often be somewhat arbitrary. In other words, many HRGs have been found to be very good surrogates for each other. One way of assessing whether particular HRGs will serve well in the methods and compositions of the invention is by assessing their correlation with the mean expression of HRGs (e.g., all known HRGs, a specific set of HRGs, etc.). Those HRGs that correlate particularly well with the mean are expected to perform well in assays of the invention, e.g., because these will reduce noise in the assay. Rankings of select HRGs according to their correlation with the mean HRG expression are given in Tables 5, 6, 7, 10, 11, 12, 13, 14, and 15. Thus, in some embodiments of each of the various aspects of the invention the plurality of test genes comprises the top 2, 3, 4, 5, 6, 7, 8, 9, 10, 11, 12, 13, 14, 15, 20, 25, 30, 35, 40 or more HRGs listed in any of Tables 5, 6, 7, 10, 11, 12, 13, 14, and 15.

In HRG signatures the particular HRGs analyzed are often not as important as the total number of HRGs. The number of HRGs analyzed can vary depending on many factors, e.g., technical constraints, cost considerations, the classification being made, the cancer being tested, the desired level of predictive power, etc. Increasing the number of HRGs analyzed in a panel according to the invention is, as a general matter, advantageous because, e.g., a larger pool of genes to be analyzed means less “noise” caused by outliers and less chance of an error in measurement or analysis throwing off the overall predictive power of the test. However, cost and other considerations will sometimes limit this number and finding the optimal number of HRGs for a signature is desirable.

To the extent measuring HRGs measures the phenomenon of hypoxia in a patient's tumor and the response of tumor cells to such hypoxia, the predictive power of a HRG signature may often cease to increase significantly beyond a certain number of HRGs. More specifically, the optimal number of HRGs in a signature (n_{O}) can be found wherever the following is true

(Pₙ₊₁−Pₙ)≦C_{O},

wherein P is the predictive power (i.e., Pₙ is the predictive power of a signature/panel with n genes and Pₙ₊₁ is the predictive power of a signature with n genes plus one) and C_{O} is some optimization constant. Predictive power can be defined in many ways known to those skilled in the art including, but not limited to, the signature's p-value. C_{O} can be chosen by the artisan based on his or her specific constraints. For example, if cost is not a critical factor and extremely high levels of sensitivity and specificity are desired, C_{O} can be set very low such that only trivial increases in predictive power are disregarded. On the other hand, if cost is decisive and moderate levels of sensitivity and specificity are acceptable, C_{O} can be set higher such that only significant increases in predictive power warrant increasing the number of genes in the signature.

Alternatively, a graph of predictive power as a function of gene number may be plotted and the second derivative of this plot taken. The point at which the second derivative decreases to some predetermined value (C_{O}′) may be the optimal number of genes in the signature.

It has been discovered that HRGs are particularly predictive in certain cancers. For example, panels of HRGs have been determined to be accurate in prognosing lung cancer and colon cancer.

Thus the invention provides a method comprising determining the status of a panel of biomarkers comprising at least two HRGs, wherein an abnormal status indicates a poor prognosis. In some embodiments the panel comprises at least 2 genes chosen from the group of genes in Tables 1, 2, 3, 5, 6, 7, 10, 11, 12, 13, 14, or 15. In some embodiments the panel comprises at least 10 genes chosen from the group of genes in Tables 1, 2, 3, 5, 6, 7, 10, 11, 12, 13, 14, or 15. In some embodiments the panel comprises at least 15 genes chosen from the group of genes in Tables 1, 2, 3, 5, 6, 7, 10, 11, 12, 13, 14, or 15. In some embodiments the panel comprises all of the genes in Tables 1, 2, 3, 5, 6, 7, 10, 11, 12, 13, 14, or 15. The invention also provides a method of determining the prognosis of lung cancer, comprising determining the status of a panel of biomarkers comprising at least two HRGs (e.g., at least two of the genes in Tables 1, 2, 3, 5, 6, 7, 10, 11, 12, 13, 14, or 15), wherein an abnormal status indicates a poor prognosis. The invention also provides a method of determining the prognosis of colon cancer, comprising determining the status of a panel of biomarkers comprising at least two HRGs (e.g., at least two of the genes in Tables 1, 2, 3, 5, 6, 7, 10, 11, 12, 13, 14, or 15), wherein an abnormal status indicates a poor prognosis. In some embodiments, the method comprises at least one of the following steps: (a) correlating abnormal status (e.g., increased expression) of the panel of biomarkers to poor prognosis; (b) concluding that the patient has a poor prognosis based at least in part on abnormal status (e.g., increased expression) of the panel of biomarkers; or (c) communicating that the patient has a poor prognosis based at least in part on abnormal status (e.g., increased expression) of the panel of biomarkers.

In some embodiments the panel comprises at least 3, 4, 5, 6, 7, 8, 9, 10, 15, 20, 25, 30, 35, 40, 45, 50 or more HRGs. In some embodiments the panel comprises between 5 and 100 HRGs, between 7 and 40 HRGs, between 5 and 25 HRGs, between 10 and 20 HRGs, or between 10 and 15 HRGs. In some embodiments HRGs comprise at least a certain proportion of the panel. Thus in some embodiments the panel comprises at least 25%, 30%, 40%, 50%, 60%, 70%, 75%, 80%, 85%, 90%, 95%, 96%, 97%, 98%, or 99% HRGs. In some embodiments the HRGs are chosen from the group consisting of the genes listed in Tables 1, 2, 3, 5, 6, 7, 10, 11, 12, 13, 14, or 15. In some embodiments the panel comprises at least 2 genes chosen from the group of genes in Tables 1, 2, 3, 5, 6, 7, 10, 11, 12, 13, 14, or 15. In some embodiments the panel comprises at least 10 genes chosen from the group of genes in Tables 1, 2, 3, 5, 6, 7, 10, 11, 12, 13, 14, or 15. In some embodiments the panel comprises at least 15 genes chosen from the group of genes in Tables 1, 2, 3, 5, 6, 7, 10, 11, 12, 13, 14, or 15. In some embodiments the panel comprises all of the genes in Tables 1, 2, 3, 5, 6, 7, 10, 11, 12, 13, 14, or 15.

### III. Systems, Computer-Implemented Methods, and Methods of Treatment According to the Invention

The results of any analyses according to the invention will often be communicated to physicians, genetic counselors and/or patients (or other interested parties such as researchers) in a transmittable form that can be communicated or transmitted to any of the above parties. Such a form can vary and can be tangible or intangible. The results can be embodied in descriptive statements, diagrams, photographs, charts, images or any other visual forms. For example, graphs showing expression or activity level or sequence variation information for various genes can be used in explaining the results. Diagrams showing such information for additional target gene(s) are also useful in indicating some testing results. The statements and visual forms can be recorded on a tangible medium such as papers, computer readable media such as floppy disks, compact disks, etc., or on an intangible medium, e.g., an electronic medium in the form of email or website on internet or intranet. In addition, results can also be recorded in a sound form and transmitted through any suitable medium, e.g., analog or digital cable lines, fiber optic cables, etc., via telephone, facsimile, wireless mobile phone, internet phone and the like.

Thus, the information and data on a test result can be produced anywhere in the world and transmitted to a different location. As an illustrative example, when an expression level, activity level, or sequencing (or genotyping) assay is conducted outside the United States, the information and data on a test result may be generated, cast in a transmittable form as described above, and then imported into the United States. Accordingly, the present invention also encompasses a method for producing a transmittable form of information on at least one of (a) expression level or (b) activity level for a panel of HRGs (as discussed in the various embodiments above) for at least one patient sample. The method comprises the steps of (1) determining at least one of (a) or (b) above according to methods of the present invention; and (2) embodying the result of the determining step in a transmittable form. The transmittable form is the product of such a method.

Techniques for analyzing such expression, activity, and/or sequence data (indeed any data obtained according to the invention) will often be implemented using hardware, software or a combination thereof in one or more computer systems or other processing systems capable of effectuating such analysis.

Thus one aspect of the present invention provides systems related to the above methods of the invention. In one embodiment the invention provides a system for determining gene expression in a tumor sample, comprising:
(1) a sample analyzer for determining the status in a sample of a panel of biomarkers including at least 4 HRGs, wherein the sample analyzer contains the sample, RNA from the sample and expressed from the genes in the panel of biomarkers, or DNA synthesized from said RNA;
(2) a first computer program for
   (a) receiving expression data on at least 4 test genes selected from the panel of biomarkers,
   (b) weighting the determined expression of each of the test genes with a predefined coefficient, and
   (c) combining the weighted expression to provide a test value, wherein the combined weight given to said at least 4 or 5 or 6 HRGs is at least 40% (or 50%, 60%, 70%, 80%, 90%, 95% or 100%) of the total weight given to the expression of all of said plurality of test genes; and optionally
(3) a second computer program for comparing the test value to one or more reference values each associated with a predetermined degree of risk of cancer.
   In some embodiments at least 20%, 50%, 75%, or 90% of said plurality of test genes are HRGs. In some embodiments the sample analyzer contains reagents for determining the status in the sample of said panel of biomarkers including at least 4 HRGs. In some embodiments the sample analyzer contains HRG-specific reagents as described below.

In another embodiment the invention provides a system for determining gene expression in a tumor sample, comprising: (1) a sample analyzer for determining the status of a panel of biomarkers in a tumor sample including at least 4 HRGs, wherein the sample analyzer contains the tumor sample which is from a patient identified as having lung cancer or colon cancer, RNA from the sample and expressed from the genes in the panel of biomarkers, or DNA synthesized from said RNA; (2) a first computer program for (a) receiving expression data on at least 4 test genes selected from the panel of biomarkers, (b) weighting the determined expression of each of the test genes with a predefined coefficient, and (c) combining the weighted expression to provide a test value, wherein the combined weight given to said at least 4 or 5 or 6 HRGs is at least 40% (or 50%, 60%, 70%, 80%, 90%, 95% or 100%) of the total weight given to the expression of all of said plurality of test genes; and optionally (3) a second computer program for comparing the test value to one or more reference values each associated with a predetermined degree of risk of cancer recurrence or progression of the lung cancer or colon cancer. In some embodiments at least 20%, 50%, 75%, or 90% of said plurality of test genes are HRGs. In some embodiments the system comprises a computer program for determining the patient's prognosis and/or determining (including quantifying) the patient's degree of risk of cancer recurrence or progression based at least in part on the comparison of the test value with said one or more reference values.

In some embodiments, the system further comprises a display module displaying the comparison between the test value and the one or more reference values, or displaying a result of the comparing step, or displaying the patient's prognosis and/or degree of risk of cancer recurrence or progression.

In some embodiments, the amount of RNA transcribed from the panel of genes including test genes (and/or DNA reverse transcribed therefrom) is measured in the sample. In addition, the amount of RNA of one or more housekeeping genes in the sample (and/or DNA reverse transcribed therefrom) is also measured, and used to normalize or calibrate the expression of the test genes, as described above.

In some embodiments, the plurality of test genes includes at least 2, 3 or 4 HRGs, which constitute at least 50%, 75% or 80% of the plurality of test genes, and preferably 100% of the plurality of test genes. In some embodiments, the plurality of test genes includes at least 5, 6 or 7, or at least 8 HRGs, which constitute at least 20%, 25%, 30%, 40%, 50%, 60%, 70%, 75%, 80% or 90% of the plurality of test genes, and preferably 100% of the plurality of test genes.

In some other embodiments, the plurality of test genes includes at least 8, 10, 12, 15, 20, 25 or 30 HRGs, which constitute at least 20%, 25%, 30%, 40%, 50%, 60%, 70%, 75%, 80% or 90% of the plurality of test genes, and preferably 100% of the plurality of test genes.

The sample analyzer can be any instrument useful in determining gene expression, including, e.g., a sequencing machine (e.g., Illumina HiSeg™, Ion Torrent PGM, ABI SOLiD™ sequencer, PacBio RS, Helicos Heliscope™, etc.), a real-time PCR machine (e.g., ABI 7900, Fluidigm BioMark™, etc.), a microarray instrument, etc.

The computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™—or JavaScript™-enabled browsers such as HotJava™, Microsoft™ Explorer™, or Netscape™ can be used. When active content web pages are used, they may include Java™ applets or ActiveX™ controls or other active content technologies.

The analysis function can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out HRG expression analysis as described above. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

Some embodiments of the present invention provide a system for determining whether a patient has increased likelihood of recurrence. Generally speaking, the system comprises (1) computer program for receiving, storing, and/or retrieving patient sample expression data for a plurality of test genes comprising at least 2, 3, 4, 5, 6, 7, 8, 9, 10, 12, 15, 20, 25 or 30 HRGs; (2) computer program means for querying this patient sample data; (3) computer program means for concluding whether there is an increased likelihood of progression or recurrence based at least in part on this patient sample data; and optionally (4) computer program means for outputting/displaying this conclusion. In some embodiments this means for outputting the conclusion may comprise a computer program means for informing a health care professional of the conclusion.

One example of such a system is the computer system [300] illustrated in FIG. 3. Computer system [300] may include at least one input module [330] for entering patient data into the computer system [300]. The computer system [300] may include at least one output module [324] for indicating whether a patient has an increased or decreased likelihood of response and/or indicating suggested treatments determined by the computer system [300]. Computer system [300] may include at least one memory module [303] in communication with the at least one input module [330] and the at least one output module [324].

The at least one memory module [303] may include, e.g., a removable storage drive [308], which can be in various forms, including but not limited to, a magnetic tape drive, a floppy disk drive, a VCD drive, a DVD drive, an optical disk drive, a flash memory drive, etc. The removable storage drive [308] may be compatible with a removable storage unit [310] such that it can read from and/or write to the removable storage unit [310]. Removable storage unit [310] may include a computer usable storage medium having stored therein computer-readable program codes or instructions and/or computer readable data. For example, removable storage unit [310] may store patient data. Example of removable storage unit [310] are well known in the art, including, but not limited to, floppy disks, magnetic tapes, optical disks, and the like. The at least one memory module may also include a hard disk drive [312], which can be used to store computer readable program codes or instructions, and/or computer readable data.

In addition, as shown in FIG. 3, the at least one memory module [303] may further include an interface [314] and a removable storage unit [313] that is compatible with interface [314] such that software, computer readable codes or instructions can be transferred from the removable storage unit [313] into computer system [300]. Examples of interface [314] and removable storage unit [313] pairs include, e.g., removable memory chips (e.g., EPROMs or PROMs) and sockets associated therewith, program cartridges and cartridge interface, and the like. Computer system [300] may also include a secondary memory module [318], such as random access memory (RAM).

Computer system [300] may include at least one processor module [302]. It should be understood that the at least one processor module [302] may consist of any number of devices. The at least one processor module [302] may include a data processing device, such as a microprocessor or microcontroller or a central processing unit. The at least one processor module [302] may include another logic device such as a DMA (Direct Memory Access) processor, an integrated communication processor device, a custom VLSI (Very Large Scale Integration) device or an ASIC (Application Specific Integrated Circuit) device. In addition, the at least one processor module [302] may include any other type of analog or digital circuitry that is designed to perform the processing functions described herein.

As shown in FIG. 3, in computer system [300], the at least one memory module [303], the at least one processor module [302], and secondary memory module [318] are all operably linked together through communication infrastructure [320], which may be a communications bus, system board, cross-bar, etc. Through the communication infrastructure [320], computer program codes or instructions or computer readable data can be transferred and exchanged. Input interface [323] may operably connect the at least one input module [323] to the communication infrastructure [320]. Likewise, output interface [322] may operably connect the at least one output module [324] to the communication infrastructure [320].

The at least one input module [330] may include, for example, a keyboard, mouse, touch screen, scanner, and other input devices known in the art. The at least one output module [324] may include, for example, a display screen, such as a computer monitor, TV monitor, or the touch screen of the at least one input module [330]; a printer; and audio speakers. Computer system [300] may also include, modems, communication ports, network cards such as Ethernet cards, and newly developed devices for accessing intranets or the internet.

The at least one memory module [303] may be configured for storing patient data entered via the at least one input module [330] and processed via the at least one processor module [302]. Patient data relevant to the present invention may include expression level information for an HRG. Patient data relevant to the present invention may also include clinical parameters relevant to the patient's disease (e.g., tumor size, cytology, stage, age, serum CEA, serum CA19-9, grade, adjuvant treatment, etc.). Any other patient data a physician might find useful in making treatment decisions/recommendations may also be entered into the system, including but not limited to age, gender, and race/ethnicity and lifestyle data such as diet information. Other possible types of patient data include symptoms currently or previously experienced, patient's history of illnesses, medications, and medical procedures.

The at least one memory module [303] may include a computer-implemented method stored therein. The at least one processor module [302] may be used to execute software or computer-readable instruction codes of the computer-implemented method. The computer-implemented method may be configured to, based upon the patient data, indicate whether the patient has an increased likelihood of recurrence, progression or response to any particular treatment, generate a list of possible treatments, etc.

In certain embodiments, the computer-implemented method may be configured to identify a patient as having or not having cancer or as having or not having an increased likelihood of recurrence or progression. For example, the computer-implemented method may be configured to inform a physician that a particular patient has cancer, has a quantified probability of having cancer, has an increased likelihood of recurrence, etc. Alternatively or additionally, the computer-implemented method may be configured to actually suggest a particular course of treatment based on the answers to/results for various queries.

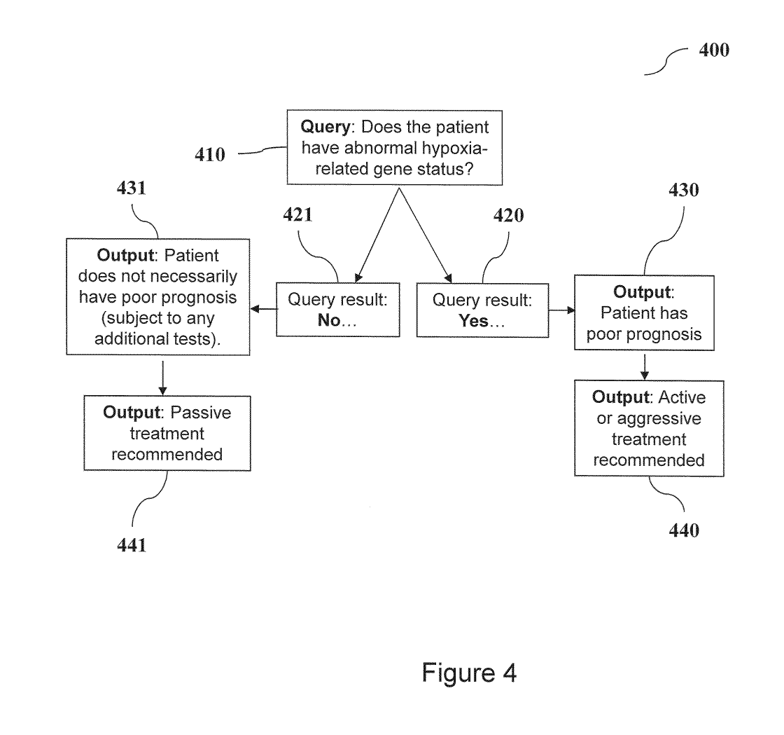

FIG. 4 illustrates one embodiment of a computer-implemented method [400] of the invention that may be implemented with the computer system [300] of the invention. The method [400] begins with a query [410]. If the answer to/result for this query is “Yes” [420], the method concludes [430] that the patient has a poor prognosis. If the answer to/result for this queries is “No” [421], the method concludes [431] that the patient does not necessarily have poor prognosis (subject to any additional tests/queries that may be desirable to be run). The method [400] may then proceed with more queries, make a particular treatment recommendation ([440], [441]), or simply end.

In some embodiments, the computer-implemented method of the invention [400] is open-ended. In other words, the apparent first step [410] in FIG. 4 may actually form part of a larger process and, within this larger process, need not be the first step/query. Additional steps may also be added onto the core methods discussed above. These additional steps include, but are not limited to, informing a health care professional (or the patient itself) of the conclusion reached; combining the conclusion reached by the illustrated method [400] with other facts or conclusions to reach some additional or refined conclusion regarding the patient's diagnosis, prognosis, treatment, etc.; making a recommendation for treatment (e.g., “patient should/should not undergo radical prostatectomy”); additional queries about additional biomarkers, clinical parameters, or other useful patient information (e.g., age at diagnosis, general patient health, etc.).

Regarding the above computer-implemented method [400], the answers to the queries may be determined by the method instituting a search of patient data for the answer. For example, to answer the query [410], patient data may be searched for HRG expression information. If such a comparison has not already been performed, the method may compare these data to some reference in order to determine if the patient has abnormal (e.g., elevated, low, negative) HRG expression. Additionally or alternatively, the method may present the query [410] to a user (e.g., a physician) of the computer system [300]. For example, the question [410] may be presented via an output module [324]. The user may then answer “Yes” or “No” via an input module [330]. The method may then proceed based upon the answer received. Likewise, the conclusions [430, 431] may be presented to a user of the computer-implemented method via an output module [324].

Thus in some embodiments the invention provides a method comprising: accessing information on a patient's HRG status stored in a computer-readable medium; querying this information to determine whether a sample obtained from the patient shows increased expression of at least one HRG; outputting [or displaying] the sample's HRG expression status. As used herein in the context of computer-implemented embodiments of the invention, “displaying” means communicating any information by any sensory means. Examples include, but are not limited to, visual displays, e.g., on a computer screen or on a sheet of paper printed at the command of the computer, and auditory displays, e.g., computer generated or recorded auditory expression of a patient's genotype.

Thus in some embodiments the invention provides a method comprising: accessing information on a patient's HRG expression stored in a computer-readable medium; querying this information to determine whether a sample obtained from the patient shows increased expression of a plurality of HRGs; and outputting [or displaying] the sample's HRG expression status. As used herein in the context of computer-implemented embodiments of the invention, “displaying” means communicating any information by any sensory means. Examples include, but are not limited to, visual displays, e.g., on a computer screen or on a sheet of paper printed at the command of the computer, and auditory displays, e.g., computer generated or recorded auditory expression of a patient's genotype.

As discussed at length above, elevated HRG expression indicates a poor prognosis (e.g., significantly increased likelihood of recurrence). Thus some embodiments provide a computer-implemented method of prognosing colorectal cancer comprising accessing information on a patient's HRG expression (e.g., from a tumor sample obtained from the patient) stored in a computer-readable medium; querying this information to determine whether the sample shows increased expression of a plurality of HRGs; and outputting (or displaying) an indication that the patient has a poor prognosis (e.g., an increased likelihood of recurrence) if the sample shows increased HRG expression. Some embodiments further comprise displaying the HRGs queried and their status (including, e.g., expression levels), optionally together with an indication of whether the HRG status indicates poor prognosis.

The practice of the present invention may also employ conventional biology methods, software and systems. Computer software products of the invention typically include computer readable media having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. Basic computational biology methods are described in, for example, Setubal et al., INTRODUCTION TO COMPUTATIONAL BIOLOGY METHODS (PWS Publishing Company, Boston, 1997); Salzberg et al. (Ed.), COMPUTATIONAL METHODS IN MOLECULAR BIOLOGY, (Elsevier, Amsterdam, 1998); Rashidi & Buehler, BIOINFORMATICS BASICS: APPLICATION IN BIOLOGICAL SCIENCE AND MEDICINE (CRC Press, London, 2000); and Ouelette & Bzevanis, BIOINFORMATICS: A PRACTICAL GUIDE FOR ANALYSIS OF GENE AND PROTEINS (Wiley & Sons, Inc., 2^{nd} ed., 2001); see also, U.S. Pat. No. 6,420,108.

The present invention may also make use of various computer program products and software for a variety of purposes, such as probe design, management of data, analysis, and instrument operation. See U.S. Pat. Nos. 5,593,839; 5,795,716; 5,733,729; 5,974,164; 6,066,454; 6,090,555; 6,185,561; 6,188,783; 6,223,127; 6,229,911 and 6,308,170. Additionally, the present invention may have embodiments that include methods for providing genetic information over networks such as the Internet as shown in U.S. Ser. No. 10/197,621 (U.S. Pub. No. 20030097222); Ser. No. 10/063,559 (U.S. Pub. No. 20020183936), Ser. No. 10/065,856 (U.S. Pub. No. 20030100995); Ser. No. 10/065,868 (U.S. Pub. No. 20030120432); Ser. No. 10/423,403 (U.S. Pub. No. 20040049354).

In one aspect, the present invention provides methods of treating a cancer patient comprising obtaining HRG expression information (e.g., the HRGs in Tables 1, 2, 3, 5, 6, 7, 10, 11, 12, 13, 14, or 15), and recommending, prescribing or administering a treatment for the cancer patient based on the HRG expression. For example, the invention provides a method of treating a cancer patient comprising:

(1) determining the expression of a plurality of HRGs; and

(2) recommending, prescribing or administering either
(a) an active (including aggressive) treatment based at least in part on abnormal HRG expression, or
(b) a passive (or less aggressive) treatment based at least in part on the absence of abnormal HRG expression.
   In some embodiments, determining the expression of a plurality of HRGs comprises receiving a report communicating such expression. In some embodiments this report communicates such expression in a qualitative manner (e.g., “high” or “increased”). In some embodiments this report communicates such expression indirectly by communicating a score (e.g., prognosis score, recurrence score, etc.) that incorporates such expression.

Whether a treatment is aggressive or not will generally depend on the cancer-type, the age of the patient, etc. For example, in breast cancer adjuvant chemotherapy is a common aggressive treatment given to complement the less aggressive standards of surgery and hormonal therapy. Those skilled in the art are familiar with various other aggressive and less aggressive treatments for each type of cancer. Aggressive treatments in colon cancer may include chemotherapy (e.g., FOLFOX, FOLFIRI, bevacizumab, cetuximab, etc.), radiotherapy, surgical resection (optionally accompanied by adjuvant chemotherapy), neoadjuvant chemotherapy, or radiotherapy, etc.

In one aspect, the invention provides compositions useful in the above methods. Such compositions include, but are not limited to, nucleic acid probes hybridizing to an HRG (or to any nucleic acids encoded thereby or complementary thereto); nucleic acid primers and primer pairs suitable for amplifying all or a portion of an HRG or any nucleic acids encoded thereby; antibodies binding immunologically to a polypeptide encoded by an HRG; probe sets comprising a plurality of said nucleic acid probes, nucleic acid primers, antibodies, and/or polypeptides; microarrays comprising any of these; kits comprising any of these; etc.

In some embodiments the invention provides a plurality of probes, each probe comprising an isolated oligonucleotide capable of selectively hybridizing to at least one of the genes in Tables 1, 2, 3, 5, 6, 7, 10, 11, 12, 13, 14, or 15. The terms “probe” and “oligonucleotide” (also “oligo”), when used in the context of nucleic acids, interchangeably refer to a relatively short nucleic acid fragment or sequence. The invention also provides primers useful in the methods of the invention. “Primers” are probes capable, under the right conditions and with the right companion reagents, of selectively amplifying a target nucleic acid (e.g., a target gene). In the context of nucleic acids, “probe” is used herein to encompass “primer” since primers can generally also serve as probes.

The probe can generally be of any suitable size/length. In some embodiments the probe has a length from about 8 to 200, 15 to 150, 15 to 100, 15 to 75, 15 to 60, or 20 to 55 bases in length. They can be labeled with detectable markers with any suitable detection marker including but not limited to, radioactive isotopes, fluorophores, biotin, enzymes (e.g., alkaline phosphatase), enzyme substrates, ligands and antibodies, etc. See Jablonski et al., NUCLEIC ACIDS RES. (1986) 14:6115-6128; Nguyen et al., BIOTECHNIQUES (1992) 13:116-123; Rigby et al., J. MOL. BIOL. (1977) 113:237-251. Indeed, probes may be modified in any conventional manner for various molecular biological applications. Techniques for producing and using such oligonucleotide probes are conventional in the art.

Probes according to the invention can be used in the hybridization/amplification/detection techniques discussed above (e.g., expression analysis). Thus, some embodiments of the invention comprise probe sets suitable for use in a microarray in detecting, amplifying and/or quantitating a plurality of HRGs. In some embodiments the probe sets have a certain proportion of their probes directed to HRGs—e.g., a probe set consisting of 10%, 20%, 30%, 40%, 50%, 55%, 60%, 65%, 70%, 75%, 80%, 85%, 90%, 95%, 96%, 97%, 98%, 99%, or 100% probes specific for HRGs. In some embodiments the probe set comprises probes directed to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 60, 70, 80 or more, or all, of the genes in Tables 1, 2, 3, 5, 6, 7, 10, 11, 12, 13, 14, or 15. Such probe sets can be incorporated into high-density arrays comprising 5,000, 10,000, 20,000, 50,000, 100,000, 200,000, 300,000, 400,000, 500,000, 600,000, 700,000, 800,000, 900,000, or 1,000,000 or more different probes. In other embodiments the probe sets comprise primers (e.g., primer pairs) for amplifying nucleic acids comprising at least a portion of one or more of the HRGs in Tables 1, 2, 3, 5, 6, 7, 10, 11, 12, 13, 14, or 15.

In another aspect of the present invention, a kit is provided for practicing the gene expression analysis methods or the prognosis methods of the present invention. Such kits may also be incorporated into the systems of the invention. The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit includes various components useful in determining the status of one or more HRGs and one or more housekeeping gene markers, using the above-discussed detection techniques. For example, the kit many include oligonucleotides specifically hybridizing under high stringency to RNA of the genes in Tables 1, 2, 3, 5, 6, 7, 10, 11, 12, 13, 14, or 15. Such oligonucleotides can be used as PCR™ primers in RT-PCR™ reactions, or hybridization probes. In some embodiments the kit comprises reagents (e.g., probes, primers, and or antibodies) for determining the status of a panel of biomarkers, where said panel comprises at least 25%, 30%, 40%, 50%, 60%, 75%, 80%, 90%, 95%, 99%, or 100% HRGs (e.g., HRGs in Tables 1, 2, 3, 5, 6, 7, 10, 11, 12, 13, 14, or 15). In some embodiments the kit consists of reagents (e.g., probes, primers, and or antibodies) for determining the expression level of no more than 2500 genes, wherein at least 5, 10, 15, 20, 30, 40, 50, 60, 70, 80, 90, 100, 120, 150, 200, 250, or more of these genes are HRGs (e.g., HRGs in Tables 1, 2, 3, 5, 6, 7, 10, 11, 12, 13, 14, or 15).

The oligonucleotides in the detection kit can be labeled with any suitable detection marker including but not limited to, radioactive isotopes, fluorephores, biotin, enzymes (e.g., alkaline phosphatase), enzyme substrates, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977). Alternatively, the oligonucleotides included in the kit are not labeled, and instead, one or more markers are provided in the kit so that users may label the oligonucleotides at the time of use.

In another embodiment of the invention, the detection kit contains one or more antibodies selectively immunoreactive with one or more proteins encoded by one or more HRGs. Examples include antibodies that bind immunologically to a protein encoded by a gene in Tables 1, 2, 3, 5, 6, 7, 10, 11, 12, 13, 14, or 15. Methods for producing and using such antibodies are well-known in the art.

Various other components useful in the detection techniques may also be included in the detection kit of this invention. Examples of such components include, but are not limited to, Taq polymerase, deoxyribonucleotides, dideoxyribonucleotides, other primers suitable for the amplification of a target DNA sequence, RNase A, and the like. In addition, the detection kit preferably includes instructions on using the kit for practice the prognosis method of the present invention using human samples.

### Example 1

The prognostic value of the hypoxia signature in Table 2 was determined in colorectal cancer. Two public data sets of expression in colon cancer samples were examined.

The dataset GSE17538 comprises 28 stage I, 72 stage II, 76 stage III and 56 stage IV colorectal cancer patients. Available outcome measures were cancer recurrence and disease-specific survival. The prognostic value of hypoxia score was evaluated with Cox proportional hazard analysis with source of samples and stage as additional parameters. Both recurrence and disease-specific survival were used as outcome variable. Results for the univariate and multivariate analysis can be found below.

Cancer Recurrence in Stages I, II and III GSE17538

| Variable | Univariate p value | Multivariate p value |
|---|---|---|
| Source | 0.001 | 0.02 |
| Stage | 0.002 | 0.03 |
| Hypoxia score | 0.000004 | 0.0002 |

Cancer Recurrence in Stage II

| Variable | Univariate p value | Multivariate p value |
|---|---|---|
| Source | 0.04 | 0.9 |
| Hypoxia score | 0.0007 | 0.0009 |

Disease-Specific Survival in Stages I, II and III GSE17538

| Variable | Univariate p value | Multivariate p value |
|---|---|---|
| Source | NS | NS |
| Stage | 0.002 | 0.04 |
| Hypoxia score | 0.0001 | 0.0016 |

In particular, the hypoxia score remains a highly significant predictor of outcome within the stage II patient set. Disease-specific survival depending on stage is displayed below.

Cancer Recurrence in Stages I, II and III from GSE14333, N = 226

| Variable | Univariate p value | Multivariate p value |
|---|---|---|
| Stage | 0.000006 | 0.0001 |
| Hypoxia score | 0.002 | 0.005 |

Cancer Recurrence in Stage II N = 94

| Variable | Univariate p value |
|---|---|
| Hypoxia score | 0.014 |

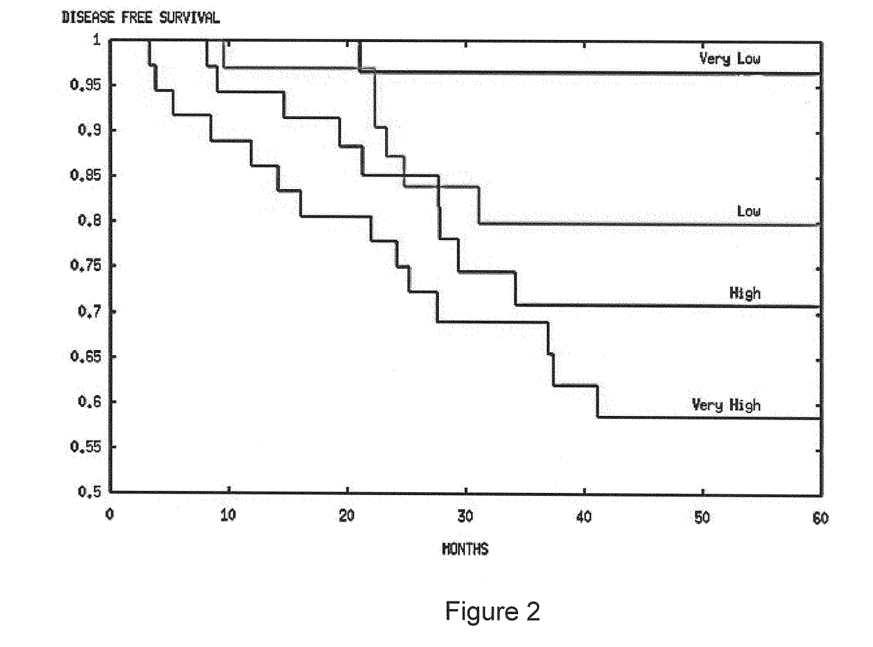

For comparison, a Kaplan-Meier plot of disease-specific survival (FIG. 2) in patients grouped by quartiles of the hypoxia score identifies a subgroup of patients with very low risk group and a subgroup with high risk group not previously seen using stage alone.

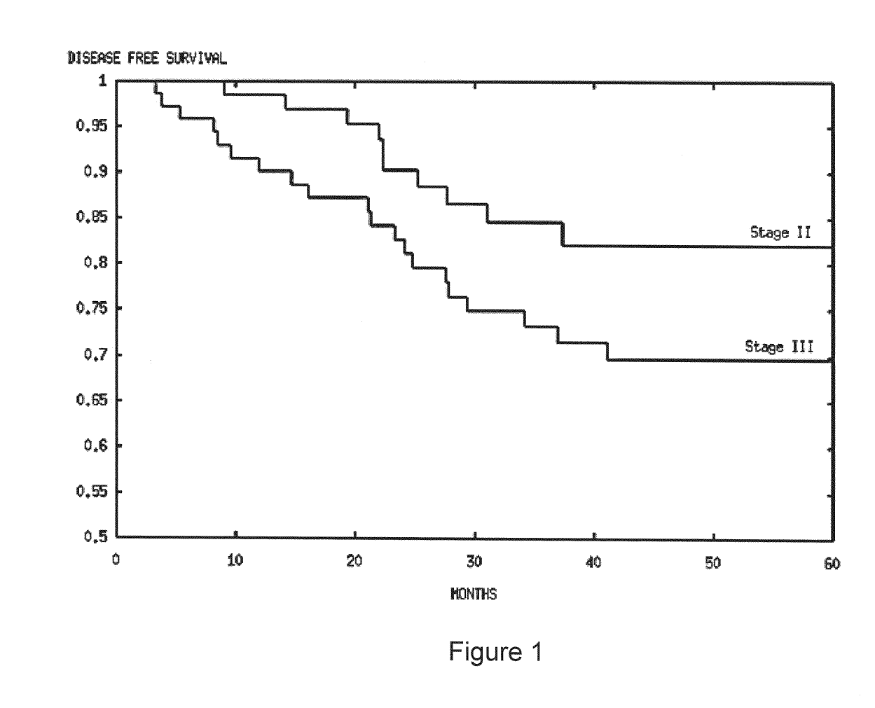

Confirmation of the predictive value of hypoxia in colon cancer was obtained from the data set GSE14333. The samples in this set have the following distribution of stages: 44 Dukes' A (=stage I), 94 Dukes' B (=stage II), 91 Dukes' C (=stage III) and 61 Dukes' D (=stage IV). The outcome variable provided is disease-free survival. P values from both univariate and multivariate Cox proportional hazard analysis are presented in FIG. 1. Both stage and hypoxia score are significant predictors of outcome in univariate analysis for stages I, II and III. Hypoxia remains a significant predictor of DFS after adjustment for stage. The hypoxia score as predictor pf outcome also remains significant when only stage II patients are included in the analysis thus supporting a hypoxia signature as an clinically useful stratification tool in Dukes' B colon cancer.

### Example 2

The prognostic value of an expression signature based on hypoxia treated genes was tested in FFPE derived RNA samples colorectal adenocarcinomas patients.

Samples

FFPE sections from 278 stage I and II colorectal cancer patients were provided by the Istituto Nazionale del Tumori in Milan. All cancers had adenocarcinoma histology. Patients who had received neoadjuvant treatment, were diagnosed as familial CRC or had higher staging were excluded. Adjuvant treatment by chemo- or radiation therapy was permitted. 43% of patients received either chemotherapy and/or radiation therapy. Outcome variables provided were progression-free survival (PFS) and overall survival (OS). Recurrence and death rates in the full cohort were 13.5% and 15%, respectively. A significant number of deaths (57%) were not preceded by disease recurrence. A third outcome variable, death with disease (DSS) was defined as death with disease recurrence to approximate disease-specific survival. For DSS patients without recurrence at the time of death were censored at the time of death.

The sample cohort was split about equally between colon cancer (48%) and rectal cancer (44%) patients, with 8% of disease localized in the border area. A higher fraction of colon cancer patients was classified with T3 stage (84%) than the rectal cancer subset (69%). Treatment choices also varied significantly between colon and rectal cancer patients. Only 33% of colon cancer patients received some form of adjuvant treatment, yet 50% of rectal cancer patients were treated. Among patients with adjuvant radiation therapy, 90% had rectal cancer and less than 2% had colon cancer.

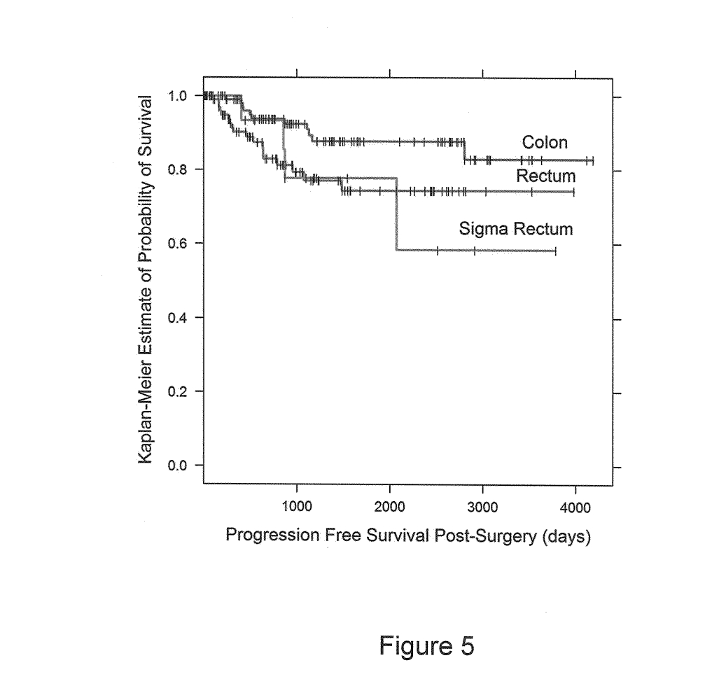

Despite lower T staging and more frequent adjuvant treatment, the rectal cancer patients had more recurrences and a higher death rate. The statistically significant difference in outcome by subtype (p=0.023) is displayed in FIG. 5. Consequently, for association with expression markers the colon and rectal patient cohorts were analyzed separately.

### Genes

Hypoxia dependent targets were selected from a list of genes up-regulated in multiple microarray data sets measuring expression in cell culture cells as a function of oxygen pressure. From a total of 42 hypoxia genes, 28 were derived from cell culture experiments. A further 14 genes were selected for high correlation with a hypoxia signature in microarray data. Five housekeeping genes were added for normalization. GAPDH (assay id HS99999905_m1) is a technical control introduced by the manufacturer. Each gene was represented by one Taqman assay. HRGs are listed in Table 3 while housekeeping genes are listed in Table 4.

TABLE 3

| | Entrez | |
|---|---|---|
| Gene | GeneId | Assay ID |
| ACTN1 | 87 | HS00998100_m1 |
| ADM | 133 | HS00181605_m1 |
| ALDOC | 230 | HS00193059_m1 |
| ANGPT2 | 285 | HS01048042_m1 |
| ANGPTL4 | 51129 | HS01101127_m1 |
| BHLHE40 | 8553 | HS00186419_m1 |
| BNIP3 | 664 | HS00969289_m1 |
| CA9 | 768 | HS00154208_m1 |
| COL5A2 | 1290 | HS00893923_m1 |
| CTSB | 1508 | HS00947439_m1 |
| DDIT4 | 54541 | HS00430304_g1 |
| DUSP1 | 1843 | HS00610256_g1 |
| ENO1 | 2023 | HS00361415_m1 |
| ERO1L | 30001 | HS00205880_m1 |
| FAM13A | 10144 | HS00208453_m1 |
| FOS | 2353 | HS00170630_m1 |
| GPI | 2821 | HS00976711_m1 |
| HIG2 | 29923 | HS00203383_m1 |
| IGFBP3 | 3486 | HS00181211_m1 |
| IL8 | 3576 | HS00174103_m1 |
| LGALS1 | 3956 | HS00355202_m1 |
| LOX | 4015 | HS00184700_m1 |
| LOXL2 | 4017 | HS00158757_m1 |
| MXI1 | 4601 | HS00365651_m1 |
| NDRG1 | 10397 | HS00608389_m1 |
| P4HA1 | 5033 | HS00914594_m1 |
| PDGFB | 5155 | HS00234042_m1 |
| PGK1 | 5230 | HS99999906_m1 |
| PLAU | 5328 | HS01547054_m1 |
| PLAUR | 5329 | HS00182181_m1 |
| PLOD2 | 5352 | HS00168688_m1 |
| SERPINE1 | 5054 | HS01126606_m1 |
| SERPINH1 | 871 | HS00241844_m1 |
| SLC16A3 | 9123 | HS00358829_m1 |
| SLC2A1 | 6513 | HS00197884_m1 |
| SLC2A3 | 6515 | HS00359840_m1 |
| SLC6A8 | 6535 | HS00940515_m1 |
| STC1 | 6781 | HS00174970_m1 |
| TGFB1 | 7040 | HS00171257_m1 |
| TMEM45A | 55076 | HS01046616_m1 |
| TNFAIP6 | 7130 | HS00200180_m1 |
| VEGFA | 7422 | HS00900055_m1 |

TABLE 4

| | Entrez | |
|---|---|---|
| Gene | GeneId | Assay ID |
| CLTC | 1213 | HS00191535_m1 |
| PPP2CA | 5515 | HS00427259_m1 |
| PSMA1 | 5682 | HS00267631_m1 |
| SLC25A3 | 5250 | HS00358082_m1 |
| TXNL1 | 9352 | HS00355488_m1 |

### Methods

Gene expression was measured by quantitative PCR. Each sample RNA was converted to cDNA and pre-amplified with a pool of all 47 assays. The pre-amplified sample was diluted and re-amplified with individual assays on TLDA cards. Samples were run in duplicate. Replicates were initiated at the step of pre-amplification.

### Analysis

The mean of the housekeeping genes was used to estimate sample quality and to normalize the expression of the target genes. Good samples were defined by the housekeeper mean and used to determine the gene-specific means for centering.

Since HRGs belong to different physiological pathways, we determined the correlation of individual genes with the mean of all HRGs. Table 5 shows the correlation coefficients for individual genes with the HRG mean derived from the full cohort. When correlations were tested only among the colon cancer samples, the ranking of genes was almost identical (Table 6).

TABLE 5

| | | Correl. |
|---|---|---|
| | | w/ |
| Gene # | Gene | Mean |
| 1 | LGALS1 | 0.77 |
| 2 | ANGPTL4 | 0.77 |
| 3 | PLAU | 0.76 |
| 4 | SERPINE1 | 0.73 |
| 5 | ADM | 0.72 |
| 6 | LOXL2 | 0.72 |
| 7 | PLAUR | 0.71 |
| 8 | STC1 | 0.71 |
| 9 | PDGFB | 0.71 |
| 10 | SERPINH1 | 0.67 |
| 11 | ACTN1 | 0.67 |
| 12 | TNFAIP6 | 0.67 |
| 13 | COL5A2 | 0.65 |
| 14 | TMEM45A | 0.65 |
| 15 | DDIT4 | 0.62 |
| 16 | LOX | 0.6 |
| 17 | DUSP1 | 0.6 |
| 18 | FOS | 0.58 |
| 19 | SLC2A3 | 0.56 |
| 20 | NDRG1 | 0.56 |
| 21 | TGFB1 | 0.52 |
| 22 | VEGFA | 0.51 |
| 23 | BHLHE40 | 0.5 |
| 24 | ERO1L | 0.48 |
| 25 | P4HA1 | 0.45 |
| 26 | PGK1 | 0.44 |
| 27 | ALDOC | 0.44 |
| 28 | SLC2A1 | 0.43 |
| 29 | IGFBP3 | 0.43 |
| 30 | CTSB | 0.42 |
| 31 | SLC16A3 | 0.41 |
| 32 | HIG2 | 0.41 |
| 33 | IL8 | 0.4 |
| 34 | SLC6A8 | 0.37 |
| 35 | PLOD2 | 0.33 |
| 36 | ENO1 | 0.26 |
| 37 | BNIP3 | 0.25 |
| 38 | FAM13A | 0.23 |
| 39 | ANGPT2 | 0.22 |
| 40 | CA9 | 0.21 |
| 41 | MXI1 | 0.18 |
| 42 | GPI | 0.14 |

TABLE 6

| | | Colon |
|---|---|---|
| | | Correl. |
| | | w/ |
| Gene # | Gene | Mean |
| 1 | ANGPTL4 | 0.76 |
| 2 | LGALS1 | 0.74 |
| 3 | PLAU | 0.74 |
| 4 | PLAUR | 0.74 |
| 5 | ADM | 0.72 |
| 6 | SERPINE1 | 0.7 |
| 7 | NDRG1 | 0.69 |
| 8 | DDIT4 | 0.67 |
| 9 | LOXL2 | 0.65 |
| 10 | ACTN1 | 0.65 |
| 11 | TNFAIP6 | 0.65 |
| 12 | STC1 | 0.64 |
| 13 | TMEM45A | 0.64 |
| 14 | SERPINH1 | 0.63 |
| 15 | DUSP1 | 0.62 |
| 16 | PDGFB | 0.62 |
| 17 | COL5A2 | 0.6 |
| 18 | ERO1L | 0.58 |
| 19 | LOX | 0.57 |
| 20 | PGK1 | 0.55 |
| 21 | FOS | 0.55 |
| 22 | SLC2A1 | 0.51 |
| 23 | SLC16A3 | 0.5 |
| 24 | HIG2 | 0.49 |
| 25 | BHLHE40 | 0.48 |
| 26 | VEGFA | 0.46 |
| 27 | CTSB | 0.45 |
| 28 | IGFBP3 | 0.45 |
| 29 | ALDOC | 0.45 |
| 30 | P4HA1 | 0.44 |
| 31 | TGFB1 | 0.42 |
| 32 | SLC6A8 | 0.41 |
| 33 | ENO1 | 0.39 |
| 34 | SLC2A3 | 0.37 |
| 35 | CA9 | 0.37 |
| 36 | BNIP3 | 0.36 |
| 37 | IL8 | 0.36 |
| 38 | FAM13A | 0.26 |
| 39 | PLOD2 | 0.23 |
| 40 | GPI | 0.2 |
| 41 | MXI1 | 0.11 |
| 42 | ANGPT2 | 0.11 |

A modified hypoxia score was calculated from the 15 genes with correlation above 0.6 in the full sample set. The genes used in the modified hypoxia score are listed in Table 7. The hypoxia score (HYP) was calculated for each sample as a base 2 logarithm of the centered copy number mean for the 15 genes that correlated most strongly with the mean.

TABLE 7

| | Correlation | | Correlation |
|---|---|---|---|
| Gene | w/ Mean | Gene | w/ Mean |
| LGALS1 | 0.77 | PDGFB | 0.71 |
| ANGPTL4 | 0.77 | SERPINH1 | 0.67 |
| PLAU | 0.76 | ACTN1 | 0.67 |
| SERPINE1 | 0.73 | TNFAIP6 | 0.67 |
| ADM | 0.72 | COL5A2 | 0.65 |
| LOXL2 | 0.72 | TMEM45A | 0.65 |
| PLAUR | 0.71 | DDIT4 | 0.62 |
| STC1 | 0.71 | | |

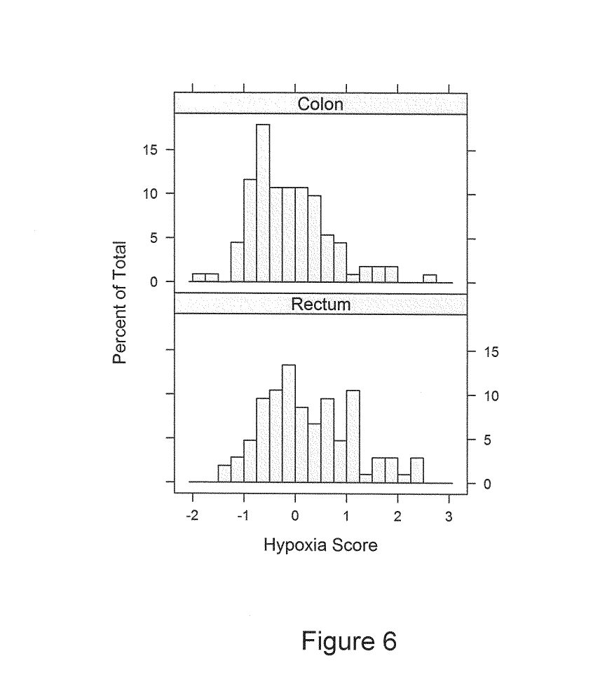

The distribution of HYP scores in colon and rectal cancer patients was very similar. A histogram of HYP scores is presented in FIG. 6.

Additional clinical variables available for analysis were stage, age, serum CEA, serum CA19-9, grade and adjuvant treatment. Only grade and tumor site were weakly associated with outcome in univariate analysis (Table 8). To account for the tumor location effect, the full cohort and the colon cancer subset were analyzed separately.

TABLE 8

| Clinical | | |
|---|---|---|
| Factor | PFS | DSS |
| Stage | 0.44 | 0.09 |
| Grade | 0.037 | 0.24 |
| Age | 0.1 | 0.04 |
| Tumor | 0.023 | 0.021 |
| Location | | |
| Adjuvant | 0.75 | 0.36 |
| Treatment | | |
| logCEA | 0.65 | 0.89 |
| logCA19.9 | 0.15 | 0.62 |

The HYP score was tested for association with progression-free survival and disease-specific survival (DSS) using Cox proportional hazard analysis. In univariate analysis, the HYP score was a significant predictor of progression-free survival in the colon cancer cohort (p=0.0091) (Table 9).

TABLE 9

| Cohort | HYP p value | N |
|---|---|---|
| Colon | 0.0091 | 97 |
| Cancer | | |
| Full cohort | 0.17 | 206 |

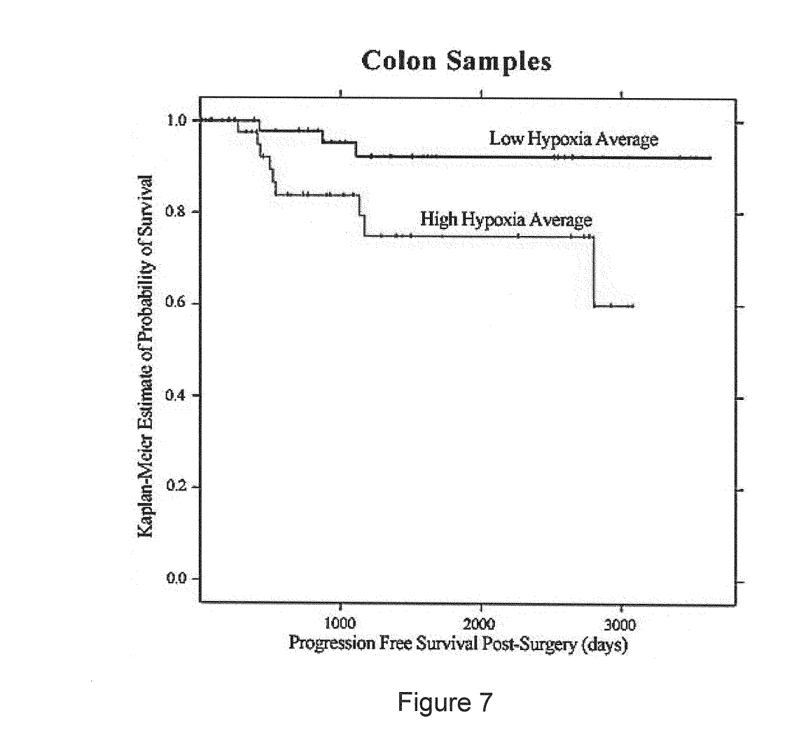

The probability of survival of patients with low and high HYP scores was estimated using the Kaplan-Meier method. The colon cancer patient cohort was separated into a low risk group with HYP scores below the mean, and a high risk group with HYP scores above the mean. The patient group with the lower HYP scores had longer progression-free survival (FIG. 7).

### Example 3

The prognostic value of an expression signature based on hypoxia treated genes was tested in FFPE derived RNA samples from lung adenocarcinoma patients.

### Samples

136 resectable, non-small cell lung cancer patients were selected from a cohort at MDA Cancer Center with at least five year follow-up period. The patients had be diagnosed with pathological stage IA, IB, IIA, or IIB and have adenocarcinoma histology. Patients who had received neoadjuvant treatment were excluded. Adjuvant treatment by chemo- or radiation therapy was permitted. Outcome variables included disease-free recurrence (DFS), overall survival (OS) and disease-specific survival (DSS). DSS was defined as death preceded by a recurrence event. Deaths not preceded by disease recurrence were censored at the time of death.

### Genes

HRGs were selected from a list of genes upregulated in multiple microarray data sets measuring expression in cell culture cells as a function of oxygen pressure. From a total of 42 hypoxia genes, 28 were derived from cell culture experiments. A further 14 genes were selected for high correlation with a hypoxia signature in microarray data. Five housekeeping genes were added for normalization. GAPDH is a technical control introduced by the manufacturer. Each gene was represented by one Taqman assay. HRGs are listed in Table 3 above while housekeeping genes are listed in Table 4 above.

### Methods

Gene expression was measured by quantitative PCR. Each sample RNA was converted to cDNA and pre-amplified with a pool of all 47 assays. The pre-amplified sample was diluted and re-amplified with individual assays on TLDA cards. Samples were run in duplicate. Replicates were initiated at the step of pre-amplification.

### Analysis

The mean of the housekeeping genes was used to estimate sample quality and to normalize the expression of the target genes. Good samples, defined as samples with a housekeeper mean of less than 21.5Ct, were used to determine the means for centering.

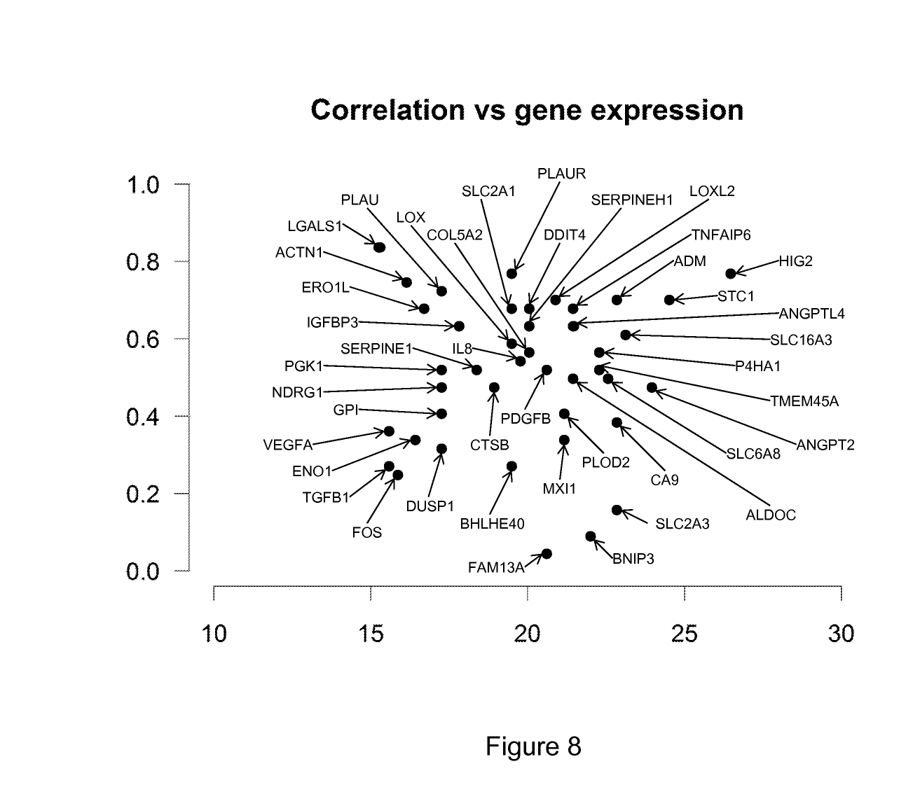

Since genes regulated in response to hypoxia belong to different physiological pathways, we determined the correlation of individual genes with the mean of all hypoxia genes. A graph showing the mean dCT of each hypoxia gene as a function of its correlation with the hypoxia mean is attached in FIG. 8. A subset of the hypoxia genes did not correlate well with the mean, irrespective of expression level. This could be due to, for example, poor performance of the chosen assay.

A modified hypoxia score was calculated from the 16 genes with correlation to the hypoxia mean of at least 0.61. The genes used in the modified hypoxia score are listed in Table 10. The hypoxia score (HYP) was calculated for each sample as a base 2 logarithm of the centered copy number mean for the 16 genes that correlated most strongly with the mean.

TABLE 10
Gene

ACTN1
ADM
ANGPTL4
DDIT4
ERO1L
HIG2
IGFBP3
LGALS1
LOXL2
PLAU
PLAUR
SERPINH1
SLC16A3
SLC2A1
STC1
TNFAIP6

The HYP score was tested for association with the three outcome measures using Cox proportional hazard analysis. In univariate analysis, the HYP score was a significant predictor of overall survival (p=0.00203) and disease-specific survival (p=0.009).

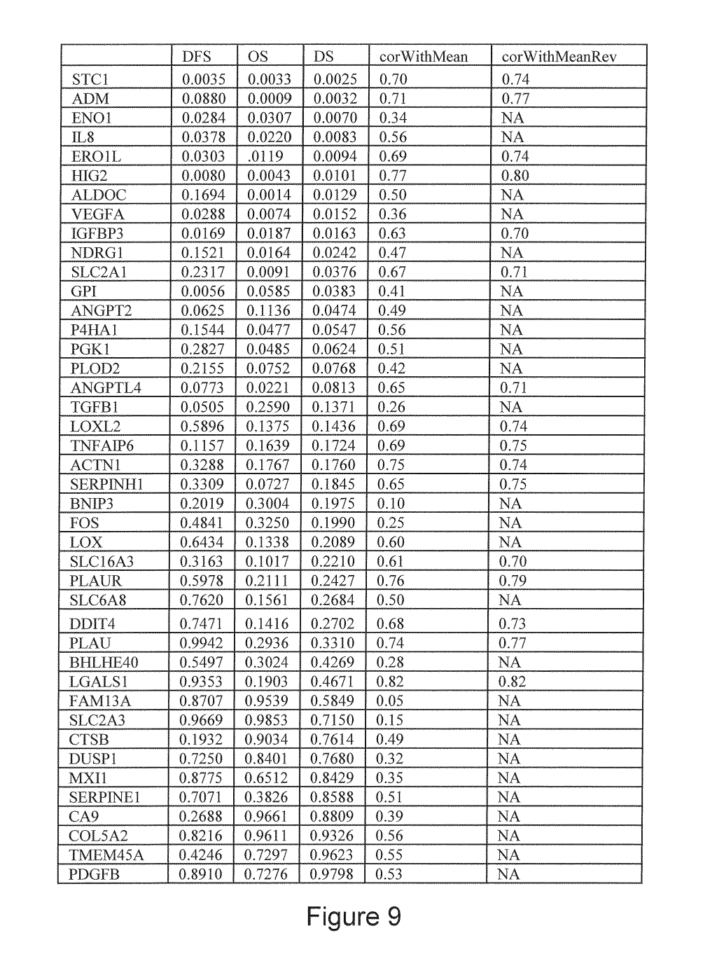

The different genes contributing to the HYP score were also tested individually for association with outcome. The results of univariate tests for each HRG in Table 3 with the three outcome measures (DFS=disease-free survival; OS=overall survival; DS=disease-specific survival) are shown in FIG. 9. Note that in cases where individual genes were not found to be significantly associated with an outcome, panels of two or more of such genes have been found to be significant. This table also lists the correlation of each gene with the hypoxia mean defined by all 42 genes (i.e., genes in Table 3) and to the mean of the 16 most correlated genes (i.e., genes in Table 10) used for association. FIG. 9 is broken out into separate tables below, with the genes in each table ranked according to either p-value or correlation to mean.

TABLE 11

| | Gene | p-value - |
|---|---|---|
| Gene # | Symbol | DFS |
| 1 | STC1 | 0.0035 |
| 2 | GPI | 0.0056 |
| 3 | HIG2 | 0.0080 |
| 4 | IGFBP3 | 0.0169 |
| 5 | ENO1 | 0.0284 |
| 6 | VEGFA | 0.0288 |
| 7 | ERO1L | 0.0303 |
| 8 | IL8 | 0.0378 |
| 9 | TGFB1 | 0.0505 |
| 10 | ANGPT2 | 0.0625 |
| 11 | ANGPTL4 | 0.0773 |
| 12 | ADM | 0.0880 |
| 13 | TNFAIP6 | 0.1157 |
| 14 | NDRG1 | 0.1521 |
| 15 | P4HA1 | 0.1544 |
| 16 | ALDOC | 0.1694 |
| 17 | CTSB | 0.1932 |
| 18 | BNIP3 | 0.2019 |
| 19 | PLOD2 | 0.2155 |
| 20 | SLC2A1 | 0.2317 |
| 21 | CA9 | 0.2688 |
| 22 | PGK1 | 0.2827 |
| 23 | SLC16A3 | 0.3163 |
| 24 | ACTN1 | 0.3288 |
| 25 | SERPINH1 | 0.3309 |
| 26 | TMEM45A | 0.4246 |
| 27 | FOS | 0.4841 |
| 28 | BHLHE40 | 0.5497 |
| 29 | LOXL2 | 0.5896 |
| 30 | PLAUR | 0.5978 |
| 31 | LOX | 0.6434 |
| 32 | SERPINE1 | 0.7071 |
| 33 | DUSP1 | 0.7250 |
| 34 | DDIT4 | 0.7471 |
| 35 | SLC6A8 | 0.7620 |
| 36 | COL5A2 | 0.8216 |
| 37 | FAM13A | 0.8707 |
| 38 | MXI1 | 0.8775 |
| 39 | PDGFB | 0.8910 |
| 40 | LGALS1 | 0.9353 |
| 41 | SLC2A3 | 0.9669 |
| 42 | PLAU | 0.9942 |

TABLE 12

| | Gene | p-value - |
|---|---|---|
| Gene # | Symbol | OS |
| 1 | ADM | 0.0009 |
| 2 | ALDOC | 0.0014 |
| 3 | STC1 | 0.0033 |
| 4 | HIG2 | 0.0043 |
| 5 | VEGFA | 0.0074 |
| 6 | SLC2A1 | 0.0091 |
| 7 | ERO1L | 0.0119 |
| 8 | NDRG1 | 0.0164 |
| 9 | IGFBP3 | 0.0187 |
| 10 | IL8 | 0.0220 |
| 11 | ANGPTL4 | 0.0221 |
| 12 | ENO1 | 0.0307 |
| 13 | P4HA1 | 0.0477 |
| 14 | PGK1 | 0.0485 |
| 15 | GPI | 0.0585 |
| 16 | SERPINH1 | 0.0727 |
| 17 | PLOD2 | 0.0752 |
| 18 | SLC16A3 | 0.1017 |
| 19 | ANGPT2 | 0.1136 |
| 20 | LOX | 0.1338 |
| 21 | LOXL2 | 0.1375 |
| 22 | DDIT4 | 0.1416 |
| 23 | SLC6A8 | 0.1561 |
| 24 | TNFAIP6 | 0.1639 |
| 25 | ACTN1 | 0.1767 |
| 26 | LGALS1 | 0.1903 |
| 27 | PLAUR | 0.2111 |
| 28 | TGFB1 | 0.2590 |
| 29 | PLAU | 0.2936 |
| 30 | BNIP3 | 0.3004 |
| 31 | BHLHE40 | 0.3024 |
| 32 | FOS | 0.3250 |
| 33 | SERPINE1 | 0.3826 |
| 34 | MXI1 | 0.6512 |
| 35 | PDGFB | 0.7276 |
| 36 | TMEM45A | 0.7297 |
| 37 | DUSP1 | 0.8401 |
| 38 | CTSB | 0.9034 |
| 39 | FAM13A | 0.9539 |
| 40 | COL5A2 | 0.9611 |
| 41 | CA9 | 0.9661 |
| 42 | SLC2A3 | 0.9853 |

TABLE 13

| | Gene | p-value - |
|---|---|---|
| Gene # | Symbol | DS |
| 1 | STC1 | 0.0025 |
| 2 | ADM | 0.0032 |
| 3 | ENO1 | 0.0070 |
| 4 | IL8 | 0.0083 |
| 5 | ERO1L | 0.0094 |
| 6 | HIG2 | 0.0101 |
| 7 | ALDOC | 0.0129 |
| 8 | VEGFA | 0.0152 |
| 9 | IGFBP3 | 0.0163 |
| 10 | NDRG1 | 0.0242 |
| 11 | SLC2A1 | 0.0376 |
| 12 | GPI | 0.0383 |
| 13 | ANGPT2 | 0.0474 |
| 14 | P4HA1 | 0.0547 |
| 15 | PGK1 | 0.0624 |
| 16 | PLOD2 | 0.0768 |
| 17 | ANGPTL4 | 0.0813 |
| 18 | TGFB1 | 0.1371 |
| 19 | LOXL2 | 0.1436 |
| 20 | TNFAIP6 | 0.1724 |
| 21 | ACTN1 | 0.1760 |
| 22 | SERPINH1 | 0.1845 |
| 23 | BNIP3 | 0.1975 |
| 24 | FOS | 0.1990 |
| 25 | LOX | 0.2089 |
| 26 | SLC16A3 | 0.2210 |
| 27 | PLAUR | 0.2427 |
| 28 | SLC6A8 | 0.2684 |
| 29 | DDIT4 | 0.2702 |
| 30 | PLAU | 0.3310 |
| 31 | BHLHE40 | 0.4269 |
| 32 | LGALS1 | 0.4671 |
| 33 | FAM13A | 0.5849 |
| 34 | SLC2A3 | 0.7150 |
| 35 | CTSB | 0.7614 |
| 36 | DUSP1 | 0.7680 |
| 37 | MXI1 | 0.8429 |
| 38 | SERPINE1 | 0.8588 |
| 39 | CA9 | 0.8809 |
| 40 | COL5A2 | 0.9326 |
| 41 | TMEM45A | 0.9623 |
| 42 | PDGFB | 0.9798 |

TABLE 14

| | | Corr. |
|---|---|---|
| | Gene | Mean - |
| Gene # | Symbol | 42 HRGs |
| 1 | LGALS1 | 0.82 |
| 2 | HIG2 | 0.77 |
| 3 | PLAUR | 0.76 |
| 4 | ACTN1 | 0.75 |
| 5 | PLAU | 0.74 |
| 6 | ADM | 0.71 |
| 7 | STC1 | 0.70 |
| 8 | ERO1L | 0.69 |
| 9 | LOXL2 | 0.69 |
| 10 | TNFAIP6 | 0.69 |
| 11 | DDIT4 | 0.68 |
| 12 | SLC2A1 | 0.67 |
| 13 | ANGPTL4 | 0.65 |
| 14 | SERPINH1 | 0.65 |
| 15 | IGFBP3 | 0.63 |
| 16 | SLC16A3 | 0.61 |
| 17 | LOX | 0.60 |
| 18 | IL8 | 0.56 |
| 19 | P4HA1 | 0.56 |
| 20 | COL5A2 | 0.56 |
| 21 | TMEM45A | 0.55 |
| 22 | PDGFB | 0.53 |
| 23 | PGK1 | 0.51 |
| 24 | SERPINE1 | 0.51 |
| 25 | ALDOC | 0.50 |
| 26 | SLC6A8 | 0.50 |
| 27 | ANGPT2 | 0.49 |
| 28 | CTSB | 0.49 |
| 29 | NDRG1 | 0.47 |
| 30 | PLOD2 | 0.42 |
| 31 | GPI | 0.41 |
| 32 | CA9 | 0.39 |
| 33 | VEGFA | 0.36 |
| 34 | MXI1 | 0.35 |
| 35 | ENO1 | 0.34 |
| 36 | DUSP1 | 0.32 |
| 37 | BHLHE40 | 0.28 |
| 38 | TGFB1 | 0.26 |
| 39 | FOS | 0.25 |
| 40 | SLC2A3 | 0.15 |
| 41 | BNIP3 | 0.10 |
| 42 | FAM13A | 0.05 |

TABLE 15

| | | Corr. |
|---|---|---|
| | Gene | Mean - |
| Gene # | Symbol | 16 HRGs |
| 1 | LGALS1 | 0.82 |
| 2 | HIG2 | 0.80 |
| 3 | PLAUR | 0.79 |
| 4 | ADM | 0.77 |
| 5 | PLAU | 0.77 |
| 6 | TNFAIP6 | 0.75 |
| 7 | SERPINH1 | 0.75 |
| 8 | STC1 | 0.74 |
| 9 | ERO1L | 0.74 |
| 10 | LOXL2 | 0.74 |
| 11 | ACTN1 | 0.74 |
| 12 | DDIT4 | 0.73 |
| 13 | SLC2A1 | 0.71 |
| 14 | ANGPTL4 | 0.71 |
| 15 | IGFBP3 | 0.70 |
| 16 | SLC16A3 | 0.70 |

The rankings of each gene according to p-value (Tables 11, 12 & 13) and correlation to the mean (Tables 14 & 15) were used to derive three different composite rankings useful in constructing HRG oanels according to the invention. Table 16 ranks the HRGs of Table 3 according to the highest composite score incorporating each gene's (a) p-value for the three outcome measures, (b) correlation to the 42-HRG mean, and (c) correlation to the 16-HRG mean, calculated by the following formula: Full composite score for each gene=(4/(p-value in Table 13))+(2/(p-value in Table 12))+(1/(p-value in Table 11))−(2/(correlation in Table 15))+(1/(correlation in Table 14)). Table 17 ranks the HRGs of Table 3 according to the highest composite score incorporating each gene's p-value for the three outcome measures, calculated by the following formula: P-value composite score for each gene=(4/(p-value in Table 13))+(2/(p-value in Table 12))+(1/(p-value in Table 11)). Table 18 ranks the HRGs of Table 3 according to the highest composite score incorporating each gene's (a) correlation to the 42-HRG mean and (b) correlation to the 16-HRG mean, calculated by the following formula: Correlation composite score for each gene=(2/(correlation in Table 15))+(1/(correlation in Table 14)). Note that for each gene in Table 3 not ranked in Table 15, a correlation of 0.10 was assigned for the purposes of calculating the composite scores.

TABLE 16

| | Gene |
|---|---|
| Gene # | Symbol |
| 1 | ADM |
| 2 | STC1 |
| 3 | ALDOC |
| 4 | HIG2 |
| 5 | ENO1 |
| 6 | ERO1L |
| 7 | IL8 |
| 8 | VEGFA |
| 9 | IGFBP3 |
| 10 | SLC2A1 |
| 11 | GPI |
| 12 | NDRG1 |
| 13 | ANGPTL4 |
| 14 | P4HA1 |
| 15 | ANGPT2 |
| 16 | PGK1 |
| 17 | PLOD2 |
| 18 | SERPINH1 |
| 19 | LOXL2 |
| 20 | TNFAIP6 |
| 21 | SLC16A3 |
| 22 | ACTN1 |
| 23 | TGFB1 |
| 24 | DDIT4 |
| 25 | PLAUR |
| 26 | LGALS1 |
| 27 | PLAU |
| 28 | LOX |
| 29 | SLC6A8 |
| 30 | FOS |
| 31 | BNIP3 |
| 32 | BHLHE40 |
| 33 | CTSB |
| 34 | SERPINE1 |
| 35 | CA9 |
| 36 | TMEM45A |
| 37 | MXI1 |
| 38 | PDGFB |
| 39 | DUSP1 |
| 40 | COL5A2 |
| 41 | SLC2A3 |
| 42 | FAM13A |

TABLE 17

| | Gene |
|---|---|
| Gene # | Symbol |
| 1 | ADM |
| 2 | STC1 |
| 3 | ALDOC |
| 4 | HIG2 |
| 5 | ENO1 |
| 6 | ERO1L |
| 7 | IL8 |
| 8 | VEGFA |
| 9 | IGFBP3 |
| 10 | SLC2A1 |
| 11 | GPI |
| 12 | NDRG1 |
| 13 | ANGPTL4 |
| 14 | P4HA1 |
| 15 | ANGPT2 |
| 16 | PGK1 |
| 17 | PLOD2 |
| 18 | TGFB1 |
| 19 | SERPINH1 |
| 20 | LOXL2 |
| 21 | TNFAIP6 |
| 22 | SLC16A3 |
| 23 | ACTN1 |
| 24 | LOX |
| 25 | BNIP3 |
| 26 | DDIT4 |
| 27 | SLC6A8 |
| 28 | FOS |
| 29 | PLAUR |
| 30 | LGALS1 |
| 31 | PLAU |
| 32 | BHLHE40 |
| 33 | CTSB |
| 34 | SERPINE1 |
| 35 | CA9 |
| 36 | FAM13A |
| 37 | TMEM45A |
| 38 | DUSP1 |
| 39 | MXI1 |
| 40 | SLC2A3 |
| 41 | PDGFB |
| 42 | COL5A2 |

TABLE 18

| | Gene |
|---|---|
| Gene # | Symbol |
| 1 | FAM13A |
| 2 | BNIP3 |
| 3 | SLC2A3 |
| 4 | FOS |
| 5 | TGFB1 |
| 6 | BHLHE40 |
| 7 | DUSP1 |
| 8 | ENO1 |
| 9 | MXI1 |
| 10 | VEGFA |
| 11 | CA9 |
| 12 | GPI |
| 13 | PLOD2 |
| 14 | NDRG1 |
| 15 | ANGPT2 |
| 16 | CTSB |
| 17 | ALDOC |
| 18 | SLC6A8 |
| 19 | PGK1 |
| 20 | SERPINE1 |
| 21 | PDGFB |
| 22 | TMEM45A |
| 23 | COL5A2 |
| 24 | IL8 |
| 25 | P4HA1 |
| 26 | LOX |
| 27 | SLC16A3 |
| 28 | IGFBP3 |
| 29 | ANGPTL4 |
| 30 | SLC2A1 |
| 31 | DDIT4 |
| 32 | SERPINH1 |
| 33 | ERO1L |
| 34 | LOXL2 |
| 35 | STC1 |
| 36 | TNFAIP6 |
| 37 | ACTN1 |
| 38 | ADM |
| 39 | PLAU |
| 40 | PLAUR |
| 41 | HIG2 |
| 42 | LGALS1 |

### Example 4

The cohort of colorectal patients from Example 2 above was enhanced by the addition of additional recurrences to improve the statistical power of the data set. 22 tumor samples of patients with early stage colorectal cancer who experienced recurrences were selected from a sample set consecutive to the one previously analyzed. Expression data for the additional recurrent samples were obtained as described in Example 2.

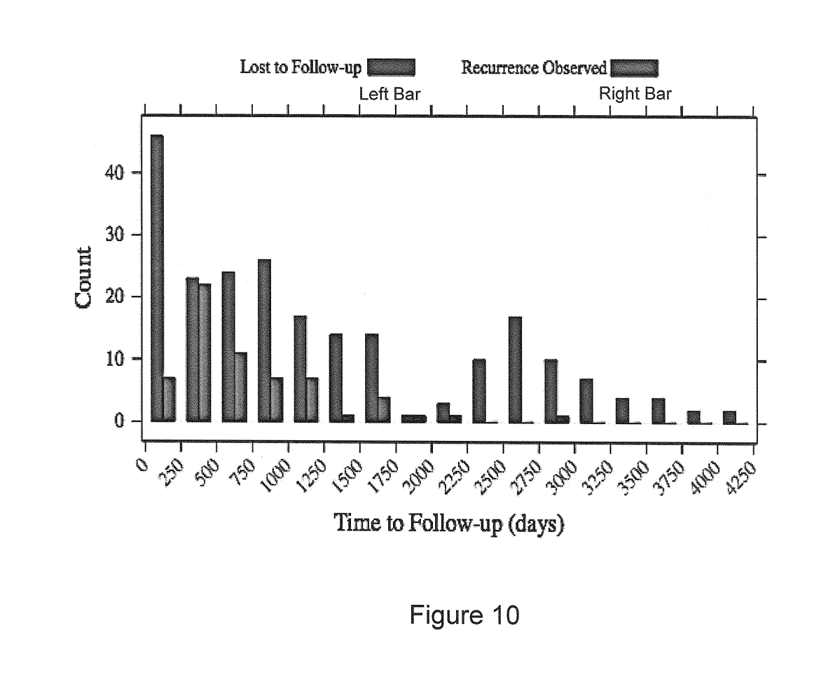

Of the total 318 samples, 286 had time to recurrence data and 293 had overall survival outcome. A plot of the time to follow-up for all samples showed a bimodal distribution. Using a threshold of 1800 days of follow-up, a binary recurrence variable was created which defined 59 patients with recurrence within 1800 days as recurrences and 60 patients lost to follow-up after 1800 days as no recurrences (FIG. 10).

A hypoxia score was calculated as the average deltaCT of the genes in Table 19. These genes were chosen by deriving the hypoxia mean expression, as described above in Example 2, for this augmented set of samples. The mean and each gene's correlation to that mean were determined both for the full set (Table 20) and for colon samples alone (Table 21). 262 patients with no missing values received a hypoxia score.

TABLE 19

| Gene # | Gene |
|---|---|
| 1 | ANGPTL4 |
| 2 | ADM |
| 3 | PDGFB |
| 4 | STC1 |
| 5 | DDIT4 |
| 6 | SERPINE1 |
| 7 | LOXL2 |
| 8 | NDRG1 |
| 9 | FOS |
| 10 | DUSP1 |
| 11 | TMEM45A |

TABLE 20

| | | Correl. |
|---|---|---|
| | | w/ |
| Gene # | Gene | Mean |
| 1 | ANGPTL4 | 0.78 |
| 2 | ADM | 0.77 |
| 3 | LGALS1 | 0.71 |
| 4 | PLAU | 0.70 |
| 5 | PDGFB | 0.69 |
| 6 | STC1 | 0.69 |
| 7 | PLAUR | 0.69 |
| 8 | DDIT4 | 0.68 |
| 9 | SERPINE1 | 0.67 |
| 10 | LOXL2 | 0.66 |
| 11 | NDRG1 | 0.66 |
| 12 | SERPINH1 | 0.65 |
| 13 | ACTN1 | 0.65 |
| 14 | FOS | 0.62 |
| 15 | DUSP1 | 0.61 |
| 16 | TMEM45A | 0.61 |
| 17 | TNFAIP6 | 0.57 |
| 18 | COL5A2 | 0.55 |
| 19 | ERO1L | 0.54 |
| 20 | VEGFA | 0.52 |
| 21 | BHLHE40 | 0.50 |
| 22 | SLC2A3 | 0.49 |
| 23 | LOX | 0.48 |
| 24 | SLC16A3 | 0.48 |
| 25 | ALDOC | 0.48 |
| 26 | SLC2A1 | 0.47 |
| 27 | P4HA1 | 0.46 |
| 28 | HIG2 | 0.46 |
| 29 | SLC6A8 | 0.45 |
| 30 | PGK1 | 0.45 |
| 31 | IGFBP3 | 0.42 |
| 32 | TGFB1 | 0.41 |
| 33 | CTSB | 0.37 |
| 34 | ENO1 | 0.32 |
| 35 | PLOD2 | 0.31 |
| 36 | IL8 | 0.30 |
| 37 | FAM13A | 0.28 |
| 38 | BNIP3 | 0.26 |
| 39 | CA9 | 0.25 |
| 40 | MXI1 | 0.22 |
| 41 | GPI | 0.19 |
| 42 | ANGPT2 | 0.13 |

TABLE 21

| | | Colon |
|---|---|---|
| | | Correl. |
| | | w/ |
| Gene # | Gene | Mean |
| 1 | ANGPTL4 | 0.76 |
| 2 | ADM | 0.75 |
| 3 | NDRG1 | 0.75 |
| 4 | PLAUR | 0.70 |
| 5 | DDIT4 | 0.69 |
| 6 | LGALS1 | 0.67 |
| 7 | PLAU | 0.66 |
| 8 | STC1 | 0.63 |
| 9 | SERPINE1 | 0.63 |
| 10 | ERO1L | 0.61 |
| 11 | ACTN1 | 0.60 |
| 12 | DUSP1 | 0.60 |
| 13 | PDGFB | 0.60 |
| 14 | SERPINH1 | 0.60 |
| 15 | PGK1 | 0.60 |
| 16 | TMEM45A | 0.58 |
| 17 | LOXL2 | 0.58 |
| 18 | FOS | 0.56 |
| 19 | HIG2 | 0.54 |
| 20 | SLC2A1 | 0.54 |
| 21 | SLC16A3 | 0.53 |
| 22 | TNFAIP6 | 0.53 |
| 23 | SLC6A8 | 0.51 |
| 24 | COL5A2 | 0.50 |
| 25 | BHLHE40 | 0.49 |
| 26 | ALDOC | 0.48 |
| 27 | VEGFA | 0.47 |
| 28 | P4HA1 | 0.45 |
| 29 | LOX | 0.44 |
| 30 | ENO1 | 0.44 |
| 31 | IGFBP3 | 0.43 |
| 32 | CA9 | 0.42 |
| 33 | BNIP3 | 0.39 |
| 34 | CTSB | 0.37 |
| 35 | FAM13A | 0.32 |
| 36 | SLC2A3 | 0.32 |
| 37 | TGFB1 | 0.31 |
| 38 | IL8 | 0.27 |
| 39 | PLOD2 | 0.25 |
| 40 | GPI | 0.23 |
| 41 | MXI1 | 0.14 |
| 42 | ANGPT2 | 0.06 |

Outcome analysis was restricted to 298 patients with stage I and stage II tumors. Higher stages were excluded. 132 patient samples were from rectal cancer, 138 were colon cancer and 27 were classified as sigma-rectum tumors. Due to the different treatments, survival was different in the three groups and each group was analyzed separately.

Associations with Outcome and Treatment in Colon Tumors:

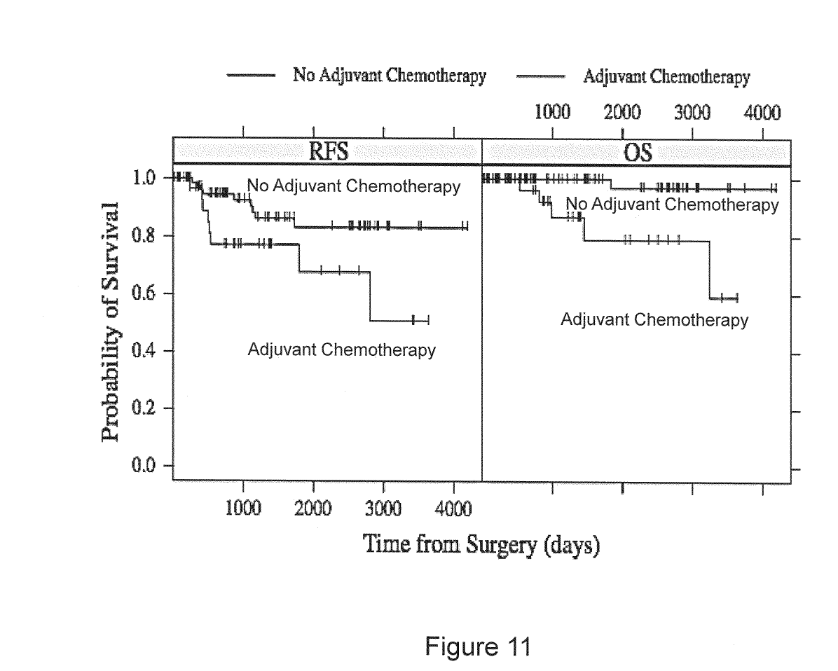

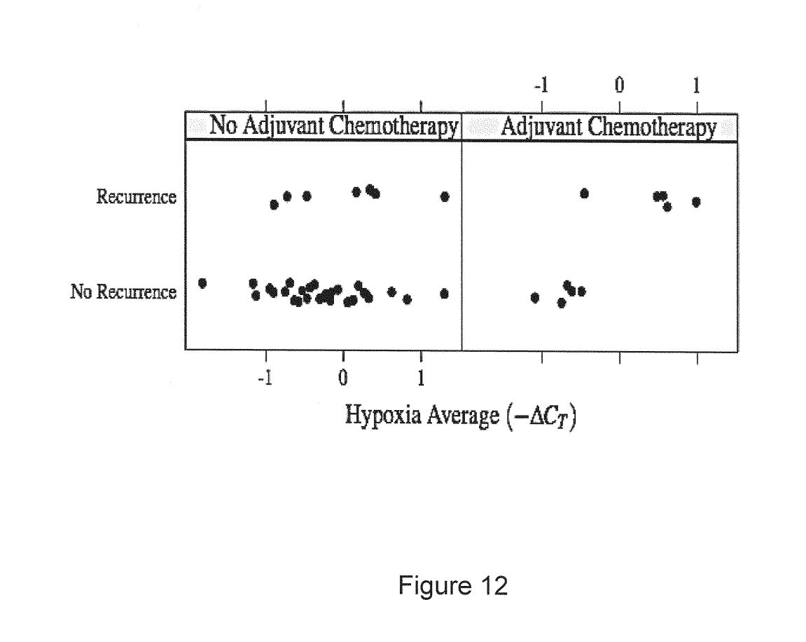

Of the clinical variables only adjuvant chemotherapy was predictive of RFS and OS with treated patients having a higher risk of recurrence (HR=2.6 (1, 6.8), p=0.053) and increased risk of death (HR=13 (1.5, 110), p=0.0046). This effect was significant for RFS. Survival curves are provided in FIG. 11. The hypoxia score was significantly associated with increased risk of recurrence (HR=2.3 (1.2, 4.3), p=0.013) and death (HR=3.3 (1, 10), p=0.05). The association between hypoxia score and outcome appeared dependent on treatment. The hazard ratio for RFS of the hypoxia average is 6.7 in patients with adjuvant treatment and 1.7 in untreated patients. Similarly, treated patients with a high hypoxia score had a worse overall survival that treated patients with a low hypoxia score. The relationship between hypoxia score and treatment is shown in FIG. 12. The interaction between hypoxia score and treatment was significant in multi-variant analysis for both RFS (p=0.031) and OS (p=0.00076).

### Example 5

In contrast to the above Examples, we have tested the prognostic ability of HRG signatures in three publicly available ER+ breast cancer cohorts: GSE2034 (n=207), GSE12093 (n=136), and GSE7390 (n=134). Cox proportional hazard analysis for distant disease recurrence was performed. There was no significant association between HRG and distant disease recurrence: p=0.40 for GSE2034, p=0.98 for GSE12093, and p=0.45 for GSE7390.

Additional studies to correlate expression of individual HRGs to the HRG expression mean were carried out on public databases as in Example 1 above. These studies yielded the following Tables showing alternate rankings according to correlation with the HRG mean.

TABLE 22

| | | | Correl. |
|---|---|---|---|
| | | | w/ |
| Gene # | Gene | EntrezID | Mean |
| 1 | ADM | 133 | 0.68 |
| 2 | LOXL2 | 4017 | 0.613 |
| 3 | LOX | 4015 | 0.612 |
| 4 | DDIT4 | 54541 | 0.602 |
| 5 | VEGFA | 7422 | 0.6 |
| 6 | SERPINE1 | 5054 | 0.597 |
| 7 | PLOD2 | 5352 | 0.578 |
| 8 | ANGPTL4 | 51129 | 0.573 |
| 9 | ERO1L | 30001 | 0.572 |
| 10 | BHLHB2 | 8553 | 0.554 |
| 11 | SLC2A3 | 6515 | 0.553 |
| 12 | LDHA | 3939 | 0.537 |
| 13 | PGK1 | 5230 | 0.534 |
| 14 | SLC2A1 | 6513 | 0.529 |
| 15 | IGFBP3 | 3486 | 0.524 |
| 16 | P4HA1 | 5033 | 0.522 |
| 17 | SLC16A3 | 9123 | 0.505 |
| 18 | ENO2 | 2026 | 0.491 |
| 19 | GAPDH | 2597 | 0.466 |
| 20 | NDRG1 | 10397 | 0.451 |
| 21 | PFKP | 5214 | 0.429 |
| 22 | TPI1 | 7167 | 0.398 |
| 23 | ALDOA | 226 | 0.384 |
| 24 | IGFBP5 | 3488 | 0.344 |
| 25 | BNIP3 | 664 | 0.338 |
| 26 | PFKFB3 | 5209 | 0.335 |
| 27 | P4HA2 | 8974 | 0.321 |
| 28 | MIF | 4282 | 0.319 |
| 29 | MXI1 | 4601 | 0.318 |
| 30 | STC2 | 8614 | 0.317 |
| 31 | TNC | 3371 | 0.276 |
| 32 | ALDOC | 230 | 0.261 |
| 33 | DUSP1 | 1843 | 0.233 |
| 34 | PDK1 | 5163 | 0.185 |
| 35 | PDGFB | 5155 | 0.17 |
| 36 | GYS1 | 2997 | 0.167 |
| 37 | ITPR1 | 3708 | 0 |
| 38 | PFKFB4 | 5210 | 0 |
| 39 | PPP1R3C | 5507 | 0 |
| 40 | PROX1 | 5629 | 0 |

TABLE 23

| | | | Correl. |
|---|---|---|---|
| | | | w/ |
| Gene # | Gene | EntrezID | Mean |
| 1 | ADM | 133 | 0.68 |
| 2 | LOXL2 | 4017 | 0.613 |
| 3 | LOX | 4015 | 0.612 |
| 4 | DDIT4 | 54541 | 0.602 |
| 5 | VEGFA | 7422 | 0.6 |
| 6 | SERPINE1 | 5054 | 0.597 |
| 7 | PLOD2 | 5352 | 0.578 |
| 8 | HIG2 | 29923 | 0.576 |
| 9 | ANGPTL4 | 51129 | 0.573 |
| 10 | ERO1L | 30001 | 0.572 |
| 11 | BHLHB2 | 8553 | 0.554 |
| 12 | SLC2A3 | 6515 | 0.553 |
| 13 | LDHA | 3939 | 0.537 |
| 14 | STC1 | 6781 | 0.537 |
| 15 | PGK1 | 5230 | 0.534 |
| 16 | SLC2A1 | 6513 | 0.529 |
| 17 | IGFBP3 | 3486 | 0.524 |
| 18 | P4HA1 | 5033 | 0.522 |
| 19 | FOSL2 | 2355 | 0.514 |
| 20 | SLC16A3 | 9123 | 0.505 |
| 21 | ENO2 | 2026 | 0.491 |
| 22 | ADFP | 123 | 0.476 |
| 23 | GAPDH | 2597 | 0.466 |
| 24 | EGLN3 | 112399 | 0.451 |
| 25 | NDRG1 | 10397 | 0.451 |
| 26 | PFKP | 5214 | 0.429 |
| 27 | JMJD6 | 23210 | 0.407 |
| 28 | TMEM45A | 55076 | 0.398 |
| 29 | TPI1 | 7167 | 0.398 |
| 30 | SLC6A8 | 6535 | 0.386 |
| 31 | ALDOA | 226 | 0.384 |
| 32 | GJA1 | 2697 | 0.374 |
| 33 | IGFBP5 | 3488 | 0.344 |
| 34 | BNIP3 | 664 | 0.338 |
| 35 | PFKFB3 | 5209 | 0.335 |
| 36 | SPAG4 | 6676 | 0.335 |
| 37 | P4HA2 | 8974 | 0.321 |
| 38 | MIF | 4282 | 0.319 |
| 39 | MXI1 | 4601 | 0.318 |
| 40 | STC2 | 8614 | 0.317 |
| 41 | TNC | 3371 | 0.276 |
| 42 | C3orf28 | 26355 | 0.274 |
| 42 | ALDOC | 230 | 0.261 |
| 43 | BNIP3L | 665 | 0.257 |
| 44 | HIST2H2BE | 8349 | 0.253 |
| 45 | CA9 | 768 | 0.243 |
| 46 | DUSP1 | 1843 | 0.233 |
| 47 | C10orf10 | 11067 | 0.229 |
| 48 | HSPA5 | 3309 | 0.207 |
| 49 | FOS | 2353 | 0.203 |
| 50 | ZFP36 | 7538 | 0.191 |
| 51 | PDK1 | 5163 | 0.185 |
| 52 | SAT1 | 6303 | 0.184 |
| 53 | FAM13A1 | 10144 | 0.179 |
| 54 | PDGFB | 5155 | 0.17 |
| 55 | GYS1 | 2997 | 0.167 |
| 56 | ZNF395 | 55893 | 0.159 |
| 57 | ADORA2B | 136 | 0.149 |
| 58 | HIST1H1C | 3006 | 0.141 |
| 59 | INHA | 3623 | 0.128 |
| 60 | INHBB | 3625 | 0.121 |
| 61 | ZFP36L2 | 678 | 0.119 |
| 62 | IGF2 | 3481 | 0.114 |
| 63 | EGFR | 1956 | 0 |
| 64 | GNB2L1 | 10399 | 0 |
| 65 | ITPR1 | 3708 | 0 |
| 66 | NR3C1 | 2908 | 0 |
| 67 | NRN1 | 51299 | 0 |
| 68 | PFKFB4 | 5210 | 0 |
| 69 | PPP1R3C | 5507 | 0 |
| 70 | PROX1 | 5629 | 0 |
| 71 | RASGRP1 | 10125 | 0 |
| 72 | RNASE4 | 6038 | 0 |
| 73 | SERPINI1 | 5274 | 0 |
| 74 | SOX9 | 6662 | 0 |
| 75 | SSR4 | 6748 | 0 |
| 76 | TFF1 | 7031 | 0 |
| 77 | APOBEC3C | 27350 | −0.184 |
| 78 | HMGCL | 3155 | −0.192 |
| 79 | ERRFI1 | 54206 | NA |
| 80 | FBXO44 | 93611 | NA |
| 81 | HLA-DRB3 | 3125 | NA |
| 82 | HOXA13 | 3209 | NA |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

1-50. (canceled)

51. A method for prognosing lung cancer, comprising:
(1) determining in a lung cancer sample the expression levels of a plurality of biomarkers comprising at least three test biomarkers selected from: ADM, STC1, ALDOC, HIG2, ENO1, ERO1L, IL8, VEGFA, IGFBP3, SLC2A1, GPI, NDRG1, ANGTPL4, P4HA1, ANGPT2, and PGK1;
(2) generating a test value by (a) weighting the determined expression levels of each biomarker in said plurality of biomarkers with a predefined coefficient, and (b) combining the weighted expressions to provide the test value, wherein the combined weight given to said at least three test biomarkers is at least 40% of the total weight given to the expression of all biomarkers in said plurality of biomarkers; and
(3)(a) diagnosing a test patient in whose sample said test value exceeds a reference value as having a lower likelihood of progression-free survival or a greater likelihood of disease progression or recurrence relative to a reference lung cancer patient with a test value equal to or lower than said reference value; or (b) diagnosing a test patient in whose sample said test value does not exceed a reference value as having a greater likelihood of progression-free survival or a lower likelihood of disease progression or recurrence relative to a reference lung cancer patient with a test value greater than said reference value.

52. The method of claim 51, wherein the plurality of biomarkers further comprises one or more additional test biomarkers selected from: PLOD2, SERPINH1, LOXL2, TNFAIP6, SLC16A3, ACTN1, TGFB1, DDIT4, PLAUR, LGALS1, PLAU, LOX, SLC6A8, FOS, BNIP3, BHLHE40, CTSB, SERPINE1, CA9, TMEM45A, MXI1, PDGFB, DUSP1, COL5A2, SLC2A3, and FAM13A.

53. The method of claim 51, wherein quantifying expression levels of the plurality of biomarkers further comprises quantifying expression levels of a plurality of housekeeping biomarkers and normalizing the quantified expression levels of the plurality of biomarkers relative to the quantified expression levels of the plurality of housekeeping biomarkers.

54. The method of claim 53, wherein the plurality of housekeeping biomarkers comprises at least one biomarker selected from: CLTC, PPP2CA, PSMA1, SLC25A3, and TXNL1.

55. The method of claim 53, wherein quantifying the expression levels of the plurality of biomarkers comprises measuring the amount of RNA for each biomarker in the lung cancer sample and quantifying the expression levels of the plurality of housekeeping biomarkers comprises measuring the amount of RNA for each housekeeping biomarker in the lung cancer sample.

56. The method of claim 51, wherein generating the test value further comprises averaging the quantified expression levels of the at least three test biomarkers.

57. The method of claim 51, wherein the lung cancer sample comprises non-small cell adenocarcinoma.

58. The method of claim 51, wherein said reference value is generated from the average test values from multiple groups of reference lung cancer patients that have been grouped based on one or more of: disease-free survival, disease-specific survival, or overall survival.

59. A method of treating lung cancer comprising:
(1) determining in a lung cancer sample the expression levels of a plurality of biomarkers comprising at least three test biomarkers selected from: ADM, STC1, ALDOC, HIG2, ENO1, ERO1L, IL8, VEGFA, IGFBP3, SLC2A1, GPI, NDRG1, ANGTPL4, P4HA1, ANGPT2, and PGK1;
(2) generating a test value by (a) weighting the determined expression levels of each biomarker in said plurality of biomarkers with a predefined coefficient, and (b) combining the weighted expressions to provide the test value, wherein the combined weight given to said at least three test biomarkers is at least 40% of the total weight given to the expression of all biomarkers in said plurality of biomarkers; and
(3)(a) administering an aggressive treatment to a test patient in whose sample said test value exceeds a reference value; or (b) administering a non-aggressive treatment to a test patient in whose sample said test value does not exceed a reference value.

60. The method of claim 59, wherein the plurality of biomarkers further comprises one or more additional test biomarkers selected from: PLOD2, SERPINH1, LOXL2, TNFAIP6, SLC16A3, ACTN1, TGFB1, DDIT4, PLAUR, LGALS1, PLAU, LOX, SLC6A8, FOS, BNIP3, BHLHE40, CTSB, SERPINE1, CA9, TMEM45A, MXI1, PDGFB, DUSP1, COL5A2, SLC2A3, and FAM13A.

61. The method of claim 59, wherein said reference value is the average test value from a group of reference lung cancer patients.

62. The method of claim 59, wherein a test value exceeding said reference value has been statistically associated, with a p-value of less than 0.05, with a lower likelihood of progression-free survival or a greater likelihood of disease progression or recurrence relative to a reference lung cancer patient with a test value equal to or lower than said reference value.

63. A system for prognosing lung cancer, comprising:
(a) a sample analyzer for quantifying in a lung cancer sample expression levels of a plurality of biomarkers comprising at least three test biomarkers selected from: ADM, STC1, ALDOC, HIG2, ENO1, ERO1L, IL8, VEGFA, IGFBP3, SLC2A1, GPI, NDRG1, ANGTPL4, P4HA1, ANGPT2, and PGK1;
(b) a first computer program for receiving expression level data quantified in (a) and generating a test value by (a) weighting the determined expression levels of each biomarker in said plurality of biomarkers with a predefined coefficient, and (b) combining the weighted expressions to provide the test value, wherein the combined weight given to said at least three test biomarkers is at least 40% of the total weight given to the expression of all biomarkers in said plurality of biomarkers;
(c) a second computer program for classifying a test patient in whose sample said test value exceeds a reference value as having a lower likelihood of progression-free survival or a greater likelihood of disease progression or recurrence relative to a reference lung cancer patient with a test value equal to or lower than said reference value; or (b) diagnosing a test patient in whose sample said test value does not exceed a reference value as having a greater likelihood of progression-free survival or a lower likelihood of disease progression or recurrence relative to a reference lung cancer patient with a test value greater than said reference value; and
(d) a display module for reporting the classification in (c).

64. The system of claim 63, wherein the plurality of biomarkers further comprises one or more additional test biomarkers selected from: PLOD2, SERPINH1, LOXL2, TNFAIP6, SLC16A3, ACTN1, TGFB1, DDIT4, PLAUR, LGALS1, PLAU, LOX, SLC6A8, FOS, BNIP3, BHLHE40, CTSB, SERPINE1, CA9, TMEM45A, MXI1, PDGFB, DUSP1, COL5A2, SLC2A3, and FAM13A.

65. A method for prognosing colorectal cancer, comprising:
(1) determining in a colorectal cancer sample the expression levels of a plurality of biomarkers comprising at least three test biomarkers selected from: ADM, STC1, ALDOC, HIG2, ENO1, ERO1L, IL8, VEGFA, IGFBP3, SLC2A1, GPI, NDRG1, ANGTPL4, P4HA1, ANGPT2, and PGK1;
(2) generating a test value by (a) weighting the determined expression levels of each biomarker in said plurality of biomarkers with a predefined coefficient, and (b) combining the weighted expressions to provide the test value, wherein the combined weight given to said at least three test biomarkers is at least 40% of the total weight given to the expression of all biomarkers in said plurality of biomarkers; and
(3)(a) diagnosing a test patient in whose sample said test value exceeds a reference value as having a lower likelihood of progression-free survival or a greater likelihood of disease progression or recurrence relative to a reference colorectal cancer patient with a test value equal to or lower than said reference value; or (b) diagnosing a test patient in whose sample said test value does not exceed a first reference value as having a greater likelihood of progression-free survival or a lower likelihood of disease progression or recurrence relative to a reference colorectal cancer patient with a test value greater than said reference value.

66. The method of claim 65, further comprising:
(4)(a) diagnosing a test patient who has been previously treated with adjuvant chemotherapy and in whose sample said test value exceeds a reference value as having a shorter predicted period of overall survival relative to a reference colorectal cancer patient with a test value equal to or lower than said reference value; or (b) diagnosing a test patient who has been previously treated with adjuvant chemotherapy and in whose sample said test value does not exceed a first reference value as having a longer predicted period of overall survival relative to a reference colorectal cancer patient with a test value greater than said reference value.

67. The method of claim 65, wherein the plurality of biomarkers further comprises one or more additional test biomarkers selected from: PLOD2, SERPINH1, LOXL2, TNFAIP6, SLC16A3, ACTN1, TGFB1, DDIT4, PLAUR, LGALS1, PLAU, LOX, SLC6A8, FOS, BNIP3, BHLHE40, CTSB, SERPINE1, CA9, TMEM45A, MXI1, PDGFB, DUSP1, COL5A2, SLC2A3, and FAM13A.

68. The method of claim 65, wherein quantifying expression levels of the plurality of biomarkers further comprises quantifying expression levels of a plurality of housekeeping biomarkers and normalizing the quantified expression levels of the plurality of biomarkers relative to the quantified expression levels of the plurality of housekeeping biomarkers.

69. The method of claim 68, wherein quantifying the expression levels of the plurality of biomarkers comprises measuring the amount of RNA for each biomarker in the lung cancer sample and quantifying the expression levels of the plurality of housekeeping biomarkers comprises measuring the amount of RNA for each housekeeping biomarker in the lung cancer sample.

70. The method of claim 65, wherein generating the test value further comprises averaging the quantified expression levels of the at least three test biomarkers.
